(19) United States
(12) Patent Application Publication
(10) Pub. No.: US 2007/0032692 A1
(43) Pub. Date: Feb. 8, 2007

(54) Catalyst and process for selective hydroconversion of normal paraffins to normal paraffin-rich lighter products

(75) Inventors: Dennis J. O'Rear, Petaluma, CA (US); Saleh A. Elomari, Fairfield, CA (US); Thomas V. Harris, Benicia, CA (US); Cong-Yan Chen, Kensington, CA (US)

Correspondence Address: CHEVRON TEXACO CORPORATION, P.O. BOX 6006, SAN RAMON, CA, 94583-0806, US

(73) Assignee: Chevron U.S.A. Inc.

(21) Appl. No.: 11/501,087

(22) Filed: Aug. 7, 2006

Related U.S. Application Data
(60) Provisional application No. 60/706,513, filed on Aug. 8, 2005.

(51) Int. Cl. C07C 4/08; C07C 4/02; C07C 6/00

(52) U.S. Cl. 585/752

(57) ABSTRACT

A process and catalyst are suitable for hydroconverting heavy normal paraffins into lighter normal paraffin products with minimal formation of isoparaffins. The process and catalyst can be used on any feed that contains heavy normal paraffins such as waxy lubricant fractions, slack wax or Fischer Tropsch products. By selectively forming a normal paraffin rich product from heavy normal paraffins, the need for normal paraffin separation and purification processes can be reduced or eliminated.

This application claims the benefit of Provisional Application 60/706,513, filed Aug. 8, 2005.

### BACKGROUND OF THE INVENTION

1. Field of the Invention

The present invention relates to a catalyst and process for hydroconverting heavy normal paraffins into lighter normal paraffin products with minimal formation of isoparaffins.

2. Background

C₅₊ liquids rich in normal paraffins are ideally suited for several applications:
Solvents, such as NORPAR™ and others
Feedstocks for ethylene production
Jet fuel and jet fuel blend components
Diesel fuel and diesel fuel blend components
Feedstock for linear alkyl benzene (LAB) production—a biodegradable solvent.
Feedstocks for isomerization to make lubricants

Historically, C₅₊ liquids rich in normal paraffins have been prepared by selectively extracting normal paraffins from mixtures, such as petroleum. This operation is relatively expensive and is limited to the content of normal paraffins in the feedstock. Normal paraffins can also be produced in the Fischer Tropsch process. However, the Fischer Tropsch process also generates heavy products that can fall outside of the range of use for the above applications. If these heavy products are converted into lighter products by hydrocracking over conventional acidic catalysts, an isoparaffin-rich product will be obtained, not a normal paraffin-rich product.

British Patent No. 2,146,350A, issued Apr. 17, 1985 describes the production of a diesel fuel with high normal paraffin content by cascading a Fisher Tropsch product to a hydrocracker. However, the high normal paraffin content in this patent is likely due to the linear hydrocarbonaceous compounds (normal paraffins, linear olefins, fatty acids, and primary linear alcohols) present in the Fischer Tropsch product that boils in the diesel range. These linear compounds are simply saturated and converted to normal paraffins. It is not evident, or likely, that there was significant conversion of heavy materials into diesel boiling range materials.

U.S. Pat. No. 5,807,413, issued Sep. 15, 1998 to Wittenbrink et al., describes a diesel engine fuel produced from Fischer-Tropsch wax by separating a light density fraction, e.g., C₅-C₁₅, preferably C₇-C₁₄ cut having at least 80+wt % n-paraffins. This patent describes how this product can be obtained directly from the Fischer Tropsch process, but does not teach how it can be obtained by hydroconversion of heavier products.

Selective cracking of heavy normal paraffins to lighter normal paraffins has been disclosed in the art. For example, in G. E. Langlois, R. F. Sullivan, and Clark J. Egan, “Hydrocracking of Paraffins with Nickel on Silica-Alumina Catalysts—the Role of Sulfiding,” Symposium on The Chemical and Physical Nature of Catalysts Presented Before the Division of the Petroleum Chemistry, American Chemical Society, Atlantic City Meeting, Sep. 12-17, 1965 (Table 1, page B-128), the conversion of n-decane (n-C₁₀) over silica alumina with different metals during hydrocracking is described.

Nickel without sulfiding gives C₄-C₇ products with low i/n ratios (0.08), but the conversion of this catalyst is very low (7.8%), and methane yields are relatively high (0.28 wt %). In comparison a sulfided nickel catalyst on the silica alumina has high conversion (52.8), and low methane yields (0.02 wt %) but gives C₄-C₇ products with high i/n ratios (6.6). Catalysts are not described that have the combination of good activity, low i/n ratio products, and low methane make.

Jule A. Rabo, “Unifying Principles in Zeolite Chemistry and Catalysis,” in Zeolites: Science and Technology, editor F. Ramoa Ribeiro et al., NATO ACS Series Vol. 80, pages 291-316, 1984 (page 295-296) discloses the use of alkali-neutralized zeolites that are free of acidic hydroxyls for cracking hexane. However, this reference discloses cracking and not hydroconversion, and significant quantities of methane (3.1 wt %) and olefins are produced.

Harry L. Coonradt and William E. Garwood, “The Mechanism of Hydrocracking,” I&EC Process Design and Development, Vol., 3 No. 1, January 1964 pages 38-45 describes the use of a platinum on silica alumina catalyst for hydrocracking hexadecane (n-C₁₆), n-heptane and n-docosane (n-C₂₀ also known as eicosane), while producing low levels of methane. Significant amount of cycloparaffins are also produced, and the product is isomerized (as noted on page 40, 1^{st} column), but the extent of isomerization is not known. While the pore properties of the support for this catalyst are not described, it probably was not microporous, as shown by the formation of cycloparaffins and isoparaffins.

B. S. Greensfelder, H. H. Voge, and G. M. Good, “Catalytic and Thermal Cracking of Pure Hydrocarbons”, Industrial and Engineering Chemistry, November 1949, pages 2573-2584, describes the evaluation of different classes of catalysts for conversion of cetane (n-C₁₆). Of particular note, activated carbon gives lower yields of methane than a thermal reaction, but still produces significant amounts of methane and also significant amounts of ethane, propane and butane. It was noted that very little chain-branching (formation of isoparaffins) was observed (page 2576, col 2, 2^{nd} paragraph). However, as with other cracking studies, significant quantities of olefins were produced, and yields of C₄— gases were excessive.

Hisatoshi Asaoka “Borosilicate synthesis in non-aqueous solvent and its activity for n-eicosane cracking”, Journal of Molecular Catalysis, 68 (1991) 301-311, discloses the preparation of a borosilicate (“Borolite-7”) which is not described as being a crystalline microporous material and apparently is amorphous. While it does show conversion of n-C₂₀ to lighter products, they are not described as being linear. The catalyst achieved partial conversion of the n-C₂₀ at 900° F. and does not contain a Group 8 metal.

Roberto Millini et al, “Synthesis and characterization of boron-containing molecular sieves” in Topics in Catalysis, 9, (1999) 13-34 present an overview of this subject. On pages 16-17 they summarize the work of Taramasso et al in making a B-ZSM-5 that contained less than 100 ppm aluminum. Pages 20-21 summarize the work of Taramasso et at in making a B-Beta that contained less than 100 ppm aluminum. The catalytic activity of the boron molecular sieves is summarized on page 32. They note that the apparent contradiction in the catalytic activity seen in early studies “ . . . was overcome when it was clearly demonstrated that trace amounts of aluminum are enough to generate remarkable catalytic activity. For instance, 80-580 ppm of Al in a B-ZSM-5 was sufficient to produce interesting results in paraffins cracking, xylene isomerization, ethylbenzene dealkylation and cyclopropane isomerization. Zeolites containing only framework boron were not active in the same reaction.” Reactions in which boron zeolites free of aluminum are claimed to be active are listed as olefin double-bond isomerization, cracking of MTBE, alkylation of aniline with methanol, and rearrangement of cyclohexanone. Hydroconversion of heavy normal paraffins to lighter normal paraffins was not described.

### SUMMARY OF THE INVENTION

In accordance with this invention, there is provided a process for converting a hydrocarbonaceous feed containing greater than 5 wt. % C₁₀₊ n-paraffins in the presence of hydrogen to produce n-paraffin products lower in molecular weight than the C₁₀₊ n-paraffins in the feed by contacting the feed under conditions comprising:

a. temperature between 600 and 800° F.,

b. pressure between 50 and 5000 psig,

c. LHSV between 0.5 and 5 with a catalyst comprising (1) a borosilicate or aluminoborosilicate molecular sieve containing at least 0.05 weight percent boron and less than 1000 ppm by weight of aluminum, or a titanosilicate molecular sieve and (2) a Group 8 metal.

The present invention also provides a process for converting a hydrocarbonaceous feed containing greater than 5 wt. % C₁₀₊ n-paraffins in the presence of hydrogen to produce n-paraffin products lower in molecular weight than the C₁₀₊ n-paraffins in the feed by contacting the feed under conditions comprising:

a. temperature between 600 and 800° F.,

b. pressure between 50 and 5000 psig,

c. LHSV between 0.5 and 5

d. conversion of n-paraffins in the feed to smaller n-paraffins of 25% to 99%

with a catalyst comprising (1) a borosilicate or aluminoborosilicate molecular sieve containing at least 0.05 weight percent boron and less than 1000 ppm by weight of aluminum, or a titanosilicate molecular sieve and (2) a Group 8 metal wherein the selectivity for the conversion of n-paraffins in the feed to smaller n-paraffins is greater than or equal to 60%. The molecular sieve can contain less than 200, for example less than 20 ppm by weight of aluminum. The Group 8 metal may be selected from the group consisting of Pt, Pd, Rh, Ir, Ru, Os and combinations thereof. Examples of molecular sieves include zeolites SSZ-13, SSZ-33, SSZ-46, SSZ-53, SSZ-55, SSZ-57, SSZ-58, SSZ-59, SSZ-60, SSZ-64, SSZ-70, ZSM-5, ZSM-11, TS-1, MTT (e.g., SSZ-32, ZSM-23 and the like) and H-Y. The hydrocarbonaceous feed can contain less than 100 ppm each of sulfur and nitrogen.

In one embodiment, the catalyst has been exposed to sulfur prior to contact with the n-paraffin-containing hydrocarbonaceous feed.

### BRIEF DESCRIPTION OF THE DRAWINGS

FIG. 1 is a schematic representation of a process for upgrading Fischer Tropsch products.

FIG. 2 is a schematic representation of a process for upgrading waxy petroleum products with lubricant base oil production.

### DETAILED DESCRIPTION OF THE INVENTION

Definitions

As used herein, the following terms have the meanings below.

Hydroconversion hydroconvert: A catalytic process which operates at pressures greater than atmospheric in the presence of hydrogen and which converts C₁₀₊ normal paraffins into lower molecular weight n-paraffins with a minimum of isomerization and without excessive formation of methane. See key features of the process as described below. Hydrotreating and hydrocracking are distinctly different catalyst processes, but also operate at pressures greater than atmospheric in the presence of hydrogen. Hydrocracking converts normal paraffins into lighter products comprising significant amounts of isoparaffins. Hydrotreating does not convert significant quantities of the feedstock to lighter products but does remove impurities. Also, in contrast, thermal cracking converts normal paraffins into lighter products with a minimum of branching, but thermal cracking does not use a catalyst. Thermal cracking typically operates at much higher temperatures, forms more methane, and makes a mixture of olefins and normal paraffins.

The current technology of n-paraffin selective hydroconversion can be differentiated from other cracking processes as shown in the next table.

| Process | Cracking | | | | |
|---|---|---|---|---|---|
| H₂ Added? | No | | Yes | | |
| Process | Thermal | Catalytic | Hydrocracking | Hydrogenolysis | n-paraffin |
| Name | | | | | Selective |
| | | | | | Hydro-conversion |
| Catalyst | None | | | | |
| Metal | None | None | Group 8 | Group 8 | Group 8 |
| Support | None | Strongly | Strongly | Non-Acidic | Weakly Acidic |
| | | Acidic | Acidic | | Molecular Sieve |
| Typical | | | | | |
| Process | | | | | |
| Conditions | | | | | |
| Temp., ° F. | >800 | >800 | <800 | 750-1100 | 600-850 |
| Pres., psig | <100 | <100 | >100 | Any | 50-5000 |
| Product | | | | | |
| Properties | | | | | |
| Paraffin i/n | <1 | >1 | >1 | <1 | <0.75 |
| | | | | | (n-C₁₆ test, C₆ |
| | | | | | product) |
| CH₄, wt % | >1 | <1 | <1 | >1 | <1 |
| Olefins - | >1% | >1% | <1% | <1% | <1% |
| Wt % and | Primary | Branched | | | |
| type | Linear | Internal | | | |

Cracking is the broadest term and refers to all processes and reactions that reduce the molecular weight of hydrocarbons by breaking them into smaller components. Two types of processes do not use added hydrogen: Thermal Cracking and Catalytic Cracking. Three types of processes use hydrogen: Hydrocracking, Hydrogenolysis, and now n-Paraffin Selective Hydroconversion.

Among other factors, n-Paraffin Selective Hydroconversion is distinguished from typical Thermal and Catalytic Cracking by the use of hydrogen and the absence of significant (1 wt % or more) amounts of olefins in the product. The catalysts and use of hydrogen are also distinguishing characteristics.

n-Paraffin Selective Hydroconversion is distinguished from typical Hydrocracking by the formation of paraffinic products having lower i/n ratios. While some past studies have shown that when a less strongly acidic hydrocracking support is used the i/n ratio decreases, these studies have not shown the low i/n ratios demonstrated in this invention, especially at high conversion levels.

n-Paraffin Selective Hydroconversion is distinguished from Hydrogenolysis by lower methane yields (lower than 1 wt. %) when compared at high conversions.

ppm values are expressed in weight unless otherwise noted.

Isoparaffin/normal paraffin ratios (i/n ratios) refer to weight ratios unless otherwise noted.

Molecular sieve is defined in Zeolite Molecular Sieves Structure Chemistry and Use by Donald W. Breck, John Wiley & Sons, pages 4-10, 1974. It includes zeolites, aluminophosphates (ALPO-11, etc), silicoaluminophosphates (SAPO-11, SM-3 etc), titanosilicates (TS-1, TS-2, SSZ-46, etc) as well as other materials.

Zeolite is defined as a molecular sieve that contains silica in the tetrahedral framework positions. Examples include, but are not limited to, silica-only (silicates), silica-alumina (aluminosilicates), silica-boron (borosilicates), and silica-titania (titanosilicates).

Weakly acidic molecular sieves are zeolites containing less than 1000 ppm of aluminum, for example less than 200 ppm or less than 20 ppm; and, when composited with Pt, Pd, Rh, Ir, Ru, Os or combinations thereof, forms an n-paraffin selective hydroconversion catalyst.

Catalysts useful in the present invention have acidities that are not as strong as Si—Al zeolites, but are stronger than the silanol groups in all-Si zeolites. Catalysts of this type are defined herein as having weak acidity.

“Weak acidity” is defined herein by calculating the Frontier Orbital Energies for bridging oxygen atoms in a series of related structures of fixed geometry (e.g. Si—O—Al, Si—O—Ga, Si—O—B, Si—O—Si) following the procedures outlined by Corma et al. in J. Am. Chem. Soc. Vol 116, No. 1, 1994 pages 136-142. The Frontier Orbital Energies are calculated from the ELUMO and EHOMO energies and are shown in Table IV of Corma et al. Table IV does not list a value for Si—O—Si, but it is separately estimated to be greater than the value of 7.44 listed in Table IV. A key point in this table is that as the acid strength increases (Si—O—B to Si—O—Al), the Frontier Orbital Energies decrease in value.

A system that is in charge balance, for example an all-silica system, has only very weak acidity and the bridging silanols have high energies, greater than 7.44 eV. Strongly acidic Si—O—Al systems have lower energies, 7.13 eV.

Materials useful in the catalysts employed in the present invention will have Frontier Orbital Energies
greater than the energy for Si—O—Al,
preferably 0.1 eV greater than then energy for Si—O—Al,
more preferably 0.25 eV greater than the energy for Si—O—Al, most preferably 0.25 eV greater than the energy for Si—O—Al and less than the energy for Si—O—Si.

Many other procedures for measuring or calculating the acidity of solids have appeared in the literature over the years. These include different calculation procedures, use of gaseous adsorbents, and change in the colors of dyes in solution. These techniques may have other uses, say in the control of the production of the catalyst, but they should not be used in place of the above method.

n-Paraffin selective hydroconversion catalyst is one that converts 80% of n-C₁₆ at conditions defined in Example 1 and temperatures <800° F., for example <700° F., or <600° F. to give a C₆ product with a i/n ratio of <0.75, for example <0.2, <0.05, or <0.01. Details are in Table 3 and in Example 1.

Pore Size and Dimensionality of Molecular Sieves Molecular sieves are crystalline materials that have regular passages (pores). If examined over several unit cells of the structure, the pores will form an axis based on the same units in the repeating crystalline structure. While the overall path of the pore will be aligned with the pore axis, within a unit cell, the pore may diverge from the axis, and it may expand in size (to form cages) or narrow. The axis of the pore is frequently parallel with one of the axes of the crystal. The narrowest position along a pore is the pore mouth. The pore size refers to the size of the pore mouth. The pore size is calculated by counting the number of tetrahedral positions that form the perimeter of the pore mouth. A pore that has 10 tetrahedral positions in its pore mouth is commonly called a 10-ring pore. Pores of relevance to catalysis in this application have pore sizes of 8 rings or greater. If a molecular sieve has only one type of relevant pore with an axis in the same orientation to the crystal structure, it is called 1-dimensional. Molecular sieves may have pores of different structures or may have pores with the same structure but oriented in more than one axis related to the crystal. In these cases, the dimensionality of the molecular sieve is determined by summing the number of relevant pores with the same structure but different axes with the number of relevant pores of different shape.

The procedure to determine the pore size and dimensionality is as follows. Table 6 is the first reference where these properties are described in the row labeled Sieve Structure. The PtB/ZSM-5 is listed as 3D 10R which means that it is a three dimensional molecular sieve with only 10 ring pores. Pt/B-SSZ-33 is listed as 12/10R 3D which means that it is a three dimensional molecular sieve with both 10 and 12 ring pores. If the structure is not listed in Table 6, the following reference is consulted: Atlas of Zeolite Structure Types, Ch. Baerlocher, W. M. Meier and D. H. Olson, 5^{th} Revised Edition, 2001. The pore size and dimensionality for different molecular sieves are in described in the Channels. This is summarized in Table 3 of this reference on pages 12-15. The number of different pores of each pore size is listed, and the dimensionality is found by summing the number of asterisks. The pore sizes are shown in bold. For example, ZSM-57 (MFS structure) is listed as
[100]10 5.1×5.4*←→[010] 8 3.3×4.8*
The dimensionality is the sum of the asterisks and is two. There are two types of pores, one 10 ring and one 8 ring. The numbers [100] and [010] refer to the orientation of these pore axes relative to the crystal axes.

If a structure is not listed in either Table 6 or the Atlas of Zeolite Structure Types, the crystal structure is determined, and the above general analysis is used to determine the pore size and dimensionality.

Microporous molecular sieves are defined as having pore mouths of 20 rings or less.

Group 8 and the Periodic Table are defined in the CRC Handbook of Chemistry and Physics, 49^{th} edition inside back cover. Group 8 refers to elements in the columns headed by Fe, Co and Ni,

Slack Wax is a by-product from lubricant oil production. A waxy 600° F.+hydrocarbonaceous material from petroleum or a synthetic source such as a Fischer Tropsch process, is dewaxed with a solvent by conventional methods to form a dewaxed lubricant base oil and a waxy slack wax by-product.

Hydrocarbonaceous feed, material or product: A pure compound or mixtures of compounds comprising H, C and optionally S, N, O and other elements. Examples include crudes, synthetic crudes, intermediate stream, petroleum products such as gasoline, jet fuel, diesel fuel, lubricant base oil, alcohols such as methanol and ethanol, etc.

Hydrocarbonaceous asset: Materials comprising H, C and optionally S, N, O and other elements used to manufacture hydrocarbonaceous products. Examples of assets are natural gas, methane, coal, petroleum, tar sands, oils shale, shale oil, waste plastics, waste tires, municipal waste, derivatives of these and mixtures.

Fischer Tropsch Process: This is described in U.S. Pat. No. 6,392,108, issued May 21, 2002 to O'Rear. It is a process that converts synthesis gas into hydrocarbonaceous products.

Syngas (or synthesis gas): A gaseous mixture containing carbon monoxide (CO) and hydrogen and optionally other components such as water and carbon dioxide. Sulfur and nitrogen and other heteroatom impurities are not desirable since they can poison the downstream Fischer Tropsch process. These impurities can be removed by conventional techniques.

Syngas Generator: (Generation of syngas is discussed in U.S. Pat. No. 6,992,114, issued Jan. 31, 2006 to O'Rear et al., which is incorporated herein by reference.) This is a process or procedure to generate synthesis gas from a hydrocarbonaceous asset. A syngas generator can be a light hydrocarbon reformer using methane or natural gas as a feedstock or a heavy hydrocarbon reformer. Reforming includes a variety of technologies such as steam reforming, partial oxidation, dry reforming, series reforming, convective reforming, and autothermal reforming. All have in common the production of syngas from methane and an oxidant (steam, oxygen, carbon dioxide, air, enriched air or combinations). The gas product typically contains some carbon dioxide and steam in addition to syngas. Series reforming, convective reforming and autothermal reforming incorporate more than one syngas-forming reaction in order to better utilize the heat of reaction. The processes for producing synthesis gas from C₁-C₃ alkanes are well known to the art. Steam reformation is typically effected by contacting C₁-C₃ alkanes with steam, preferably in the presence of a reforming catalyst, at a temperature of about 1300° F. (705° C.) to about 1675° F. (913° C.) and pressures from about 10 psia (0.7 bars) to about 500 psia (34 bars). Suitable reforming catalysts which can be used include, for example, nickel, palladium, nickel-palladium alloys, and the like. Regardless of the system used to produce syngas it is desirable to remove any sulfur compounds, e.g., hydrogen sulfide and mercaptans, contained in the C₁-C₃ alkane feed. This can be affected by passing the C₁-C₃ alkane gas through a packed bed sulfur scrubber containing zinc oxide bed or another slightly basic packing material. If the amount of C₁-C₃ alkanes exceeds the capacity of the synthesis gas unit, the surplus C₁-C₃ alkanes can be used to provide energy throughout the facility. For example, excess C₁-C₃ alkanes may be burned in a steam boiler to provide the steam used in a thermal cracking step.

In a heavy hydrocarbon reformer, the process involves converting coal, heavy petroleum stocks such as resid, or combinations thereof, into syngas. The temperature in the reaction zone of the syngas generator is about 1800° F.-3000° F. and the pressure is about 1 to 250 atmospheres. The atomic ratio of free oxygen in the oxidant to carbon in the feedstock (O/C, atom/atom) is about 0.6 to 1.5, preferably about 0.80 to 1.3. The free oxygen-containing gas or oxidant may be air, oxygen-enriched air, i.e., greater than 21 up to 95 mole % O₂ or substantially pure oxygen, i.e., greater than 95 mole % O₂. The effluent gas stream leaving the partial oxidation gas generator generally has the following composition in mole % depending on the amount and composition of the feed streams: H₂:8.0 to 60.0; CO: 8.0 to 70.0; CO₂:1.0 to 50.0, H₂:2.0 to 75.0, CH₄:0.0 to 30.0, H₂S:0.1 to 2.0, COS: 0.05 to 1.0, N₂:0.0 to 80.0, Ar:0.0 to 2.0. Particulate matter entrained in the effluent gas stream may comprise generally about 0.5 to 30 wt. % more, particularly about 1 to 10 wt. % of particulate carbon (basis weight of carbon in the feed to the gas generator). Fly ash particulate matter may be present along with the particulate carbon and molten slag. Conventional gas cleaning and/or purification steps may be employed such as that described in U.S. Pat. No. 5,423,894, issued Jun. 13, 1995 to Child et al.

The syngas can also be generated by directly converting underground hydrocarbonaceous assets. An example of a process to convert underground (or in situ) hydrocarbonaceous assets is described in U.S. Pat. No. 6,698,515, issued Mar. 2, 2004 to Karanikas et al.

Determination of Normal Paraffins in Wax Samples

Determination of normal paraffins in wax-containing samples should use a method that can determine the content of individual C₇ to C₁₁₀ n-paraffins with a limit of detection of 0.1 wt %. The preferred method used is as follows.

Quantitative analysis of normal paraffins in wax is determined by gas chromatography (GC). The GC (Agilent 6890 or 5890 with capillary split/splitless inlet and flame ionization detector) is equipped with a flame ionization detector, which is highly sensitive to hydrocarbons. The method utilized a methyl silicone capillary column, routinely used to separate hydrocarbon mixtures by boiling point. The column was fused silica, 100% methyl silicone, 30 meters length, 0.25 mm ID, 0.1 micron film thickness supplied by Supelco. Helium is the carrier gas (2 ml/min) and hydrogen and air are used as the fuel to the flame.

The wax is melted to obtain a 0.1 g homogeneous sample. The sample is immediately dissolved in carbon disulfide to give a 2 wt % solution. If necessary, the solution is heated until visually clear and free of solids, and then injected into the GC. The methyl silicone column is heated using the following temperature program:
Initial temp: 150° C. (If C₇ to C₁₅ hydrocarbons are present, the initial temperature was 50° C.)
Ramp: 6° C. per minute
Final Temp: 400° C.
Final hold: 5 minutes or until peaks no longer elute

The column then effectively separates, in the order of rising carbon number, the normal paraffins from the non-normal paraffins. A known reference standard is analyzed in the same manner to establish elution times of the specific normal-paraffin peaks. The standard used is ASTM D2887 n-paraffin standard, purchased from a vendor (Agilent or Supelco), spiked with 5 wt % Polywax 500 polyethylene (purchased from Petrolite Corporation in Oklahoma). The standard is melted prior to injection. Historical data collected from the analysis of the reference standard also guarantees the resolving efficiency of the capillary column.

If present in the sample, normal paraffin peaks are well separated and easily identifiable from other hydrocarbon types present in the sample. Those peaks eluting outside the retention time of the normal paraffins are called non-normal paraffins. The total sample is integrated using baseline hold from start to end of run. N-paraffins are skimmed from the total area and are integrated from valley to valley. All peaks detected are normalized to 100%. HP Chemstation was used for the peak identification and calculation of results.

Measurement of B, Al and Si in zeolites and solid reagents: Elemental analysis is performed by ICP-OES following digestion of the materials with HF digestion in Teflon beakers. The limit of detection in the HF solution of B is 0.1 ppb and Al is 0.2 ppb. The ICP-OES technique is described in Willard, Merritt, Dean and Settle; Instrumental Methods of Analysis, 7th edition. This translates to a limit of detection in the original material of about 10 ppm for aluminum provided sufficient material (at least 1 gram) is dissolved. The preferred method is to measure the components on the synthesized sample, which requires at least 1 gram of material. However, when the available sample size is less than 1 gram, as is the case for small research samples, it is acceptable to measure trace levels of aluminum in the components (silica reagent, boron reagent, water, etc) and calculate the maximum amount of aluminum in the sample assuming that all is present in the final product.

For this work, the aluminum content of the reagents used in this study were:

| | |
|---|---|
| Tetraethylorthosilicate (TEOS) | Not detected |
| Cab-O-Sil | 18 ppm |
| Deionized water | Not detected |
| NH₄NO₃ | Not detected |
| Na₂B₄O₇ | Described in Examples |

The aluminum content of weakly acidic molecular sieves made in this work without deliberate addition of aluminum is below 20 ppm.

Measurement of sulfur and nitrogen in feedstocks: For samples with pour points below 50° C. the nitrogen content in the samples is measured by the following procedure. A 0.2-2.0 g portion of sample is diluted using a solvent. Ten microliters of diluted sample is injected into a quartz boat sitting in a carrier gas stream. The boat is slowly moved into a combustion furnace and the diluted sample evaporates. The volatilized sample gases mix with oxygen in the hot furnace, burn, and produce, among other gases, nitric oxide (NO). The gases are dried, swept into a detection cell, and mixed with ozone. The ozone reacts with NO, giving off chemiluminescence light in the process. The light is detected and converted to a voltage, and peak areas are obtained for each sample. The areas are related to concentration by means of standards. Results are reported as ppm nitrogen. Detail test method can be found in ASTM D4629.

The sulfur content is measured by the following procedure for samples containing less than 1000 ppm sulfur and having pour points below 50° C. About 10 microliters sample is vaporized and combined with oxygen at a temperature of 1000° C. where the sulfur is oxidized to sulfur dioxide, SO₂. Water product during the sample combustion is removed and the sample combustion gases are next exposed to UV light. The SO₂ absorbs the energy from the UV light and is converted to excited SO₂*. The fluorescence emitted from the excited SO₂* as it returns to a stable state SO₂ is detected by a photomultiplier tube and the result signal is a measure of the sulfur contained in the sample. Detail test method can be found in ASTM D5453. For samples containing more than 1000 ppm sulfur, the sulfur content is measured by XRF.

For samples with pour points of 50° C. and above, especially those from a Fischer Tropsch process, the preferred method to measure heteroatom contents is described in U.S. Pat. No. 6,503,956, issued Jan. 7, 2003 to Maleksadeh.

We have discovered catalysts that can be used to hydroconvert heavy normal paraffins to form C₅₊ normal paraffins. Key features of the catalyst are:
A Group 8 metal (for example Pt), optionally exposed to sulfur.
A microporous weakly acidic molecular sieve devoid of strong acid functions, preferably devoid of the combination of silicon and aluminum. The microporous weakly acidic molecular sieve can be composed of a siliceous framework such as a zeolite, and preferably the weakly acidic molecular sieve is a zeolite and contains pores of 12-ring or less, for example pores of 10-ring or less. The weakly acidic molecular sieve contains a second oxide element (silica being the first) that does not induce strong acidity but acts to promote hydroncoversion. For example, this second oxide element is boron or titanium, most preferably boron.
A defined activity and product selectivity for conversion of n-C₁₆ at standard conditions.
It can be advantageous to expose the catalyst to sulfur prior to performing the n-paraffin hydroconversion. This pre-exposure can be done by use of hydrogen sulfide or a light sulfur-containing compound (e.g., dimethyl sulfide, dimethyl disulfide and the like). Alternatively, feedstock containing more than 1 ppm sulfur, for example more than 10 ppm sulfur, can be run over the catalyst prior to the n-paraffin hydroconversion. Then a low sulfur feed containing n-paraffins can be hydroconverted using the sulfided catalyst. Furthermore, we have found that catalysts useful for this invention can be identified by performing a simple test using n-C₁₆ as a feedstock. A catalyst comprising at least one molecular sieve and at least one Group 8 metal is tested by positioning 0.5 g of the catalyst in a ¼ inch internal diameter tube reactor. If the catalyst converts at least 80% of n-hexadecane at a temperature of ≦800° F., at a pressure of 1200 psig, in the presence of hydrogen at a flow of 160 ml/min and a n-hexadecane feed rate of 1 ml/hr and 2) produces a product comprising C₆ hydrocarbons having an iso/normal weight ratio of less than 0.75, the catalyst passes the test and is considered useful in the present invention.

Features of the n-paraffin selective hydroconversion process include:
A feedstock containing >5 wt % C₁₀₊ normal paraffins, preferably >50 wt % C₁₀₊ normal paraffins, most preferably >80 wt % C₁₀₊ normal paraffins. Suitable sources of feedstocks are derived from petroleum products and synthetic crudes. Slack waxes and Fischer Tropsch products are preferred feedstocks.
The feedstock can contain low levels of sulfur and nitrogen (see the table of preferable properties below).
The feedstock can contain low levels of oxygen, specifically less than 1 wt %, for example less than 1000 weight ppm, or less than 100 weight ppm.
The feedstock can be purified by hydrotreatment prior to hydroconversion.
The pressure is between 50 and 5000 psig, for example between 100 and 2000 psig, or at or between 250 and 1000 psig.
The LSHV is preferably between 0.5 and 5, for example between 1 and 2 LHSV.
The temperature is between 600 and 850° F., for example between 700 and 800° F., or between 725 and 775° F.
If the process is conducted in a continuous manner, the per-pass conversion of the heavy paraffins in the feedstock is between 25 and 99%, for example between 50 and 80%, or between 60 and 75%.
The C₅₊ normal paraffinic product is separated from any unconverted feedstock for example by distillation.
Performance is enhanced when the catalyst is exposed to sulfur prior to contact with the feedstock to be hydroconverted.
   Molecular Sieves

Examples of molecular sieves useful in n-Paraffin Selective Hydroconversion of the present invention include, but are not limited to, those designated SSZ-13, SSZ-33, SSZ-46, SSZ-53, SSZ-55, SSZ-57, SSZ-58, SSZ-59, SSZ-64, ZSM-5, ZSM-11, TS-1, MTT (e.g., SSZ-32, ZSM-23 and the like), H-Y, SSZ-60 and SSZ-70. It should be noted that these molecular sieves each contain silicon as the major tetrahedral element, have 8 to 12 ring pores, and are microporous molecular sieves as defined above. To be useful as n-paraffin selective hydroconversion catalysts they must contain low levels of strongly acidic components (such as aluminum), contain a weakly acidic component (such as boron or titanium) and be composited with a Group 8 metal, such as platinum.

Molecular sieve SSZ-13 is disclosed in U.S. Pat. No. 4,544,538, issued Oct. 1, 1985 to Zones.

Molecular sieve SSZ-33 is disclosed in U.S. Pat. No. 4,963,337, issued Oct. 16, 1990 to Zones.

Molecular sieve SSZ-46 is disclosed in U.S. Pat. No. 5,968,474, issued Oct. 19, 1999 to Nakagawa et al.

Molecular sieve SSZ-53 is disclosed in U.S. Pat. No. 6,632,416, issued Oct. 14, 2003 to Elomari.

Molecular sieve SSZ-55 is disclosed in U.S. Pat. No. 6,475,463, issued Nov. 5, 2002 to Elomari et al.

Molecular sieve SSZ-57 is disclosed in U.S. Pat. No. 6,544,495, issued Apr. 8, 2003 to Elomari.

Molecular sieve SSZ-58 is disclosed in U.S. Pat. No. 6,555,089, issued Apr. 29, 2003 to Elomari.

Molecular sieve SSZ-59 is disclosed in U.S. Pat. No. 6,464,956, issued Oct. 15, 2002 to Elomari.

Molecular sieve SSZ-64 is disclosed in U.S. Pat. No. 6,569,401, issued May 27, 2003 to Elomari.

Molecular sieve ZSM-5 is disclosed in U.S. Pat. No. 3,702,886, issued Nov. 14, 1972 to Argauer et al.

Molecular sieve ZSM-11 is disclosed in U.S. Pat. No. 3,709,979, issued Jan. 9, 1973 to Chu.

Molecular sieve TS-1 is disclosed in U.S. Pat. No. 4,410,501, issued Oct. 18, 1983 to Taramasso et al.

Molecular sieve MTT is disclosed in U.S. Pat. No. 5,053,373, issued Oct. 1, 1991 to Zones, U.S. Pat. No. 5,252,527, issued Oct. 12, 1993 to Zones, and U.S. Pat. No. 4,076,842, issued Feb. 8, 1978 to Plank et al.

Molecular sieve Y is disclosed in U.S. Pat. No. 3,310,007, issued Apr. 21, 1964 to Breck.

Molecular sieve SSZ-60 is disclosed in U.S. Pat. No. 6,620,401, issued Sep. 16, 2003 to Elomari and U.S. Pat. No. 6,540,906, issued Apr. 1, 2003 to Elomari.

Molecular sieve SSZ-70 is disclosed in published U.S. Patent Application No. 20060140855 A1, published Jun. 29, 2006.

Each of the foregoing patents and patent application is incorporated by reference herein in its entirety.

Catalyst Preparation

Synthesis of Microporous Weakly Acidic Molecular Sieves

Microporous weakly acidic molecular sieves can be synthesized from mixtures comprising inorganic reagents, water, optionally organic structure directing agents (typically amines), and optionally inorganic bases (NaOH, KOH, etc). This synthesis is well known in the art and is widely practiced commercially. It is typically performed in stirred autoclaves, operating at pressures above atmospheric pressure, for a few days. The product of the synthesis is recovered by filtration, centrifugation and other techniques. The microporous weakly acidic molecular sieve product is washed to remove impurities. A feature of this invention is the selection of reagents to avoid introduction of strong acid functions into the microporous weakly acidic molecular sieve. Especially, aluminum is to be avoided in the synthesis of silica-based microporous weakly acidic molecular sieves, such as borosilicates. Aluminum in water can be controlled by purification, such as ion exchange. Aluminum in the inorganic reagents can be controlled by selection of the reagent. For silica-containing reagents, the aluminum content must be as low as possible: less than 500 weight ppm, preferably less than 100 weight ppm, most preferably less than 20 weight ppm. An example of a low aluminum silica-containing reagent is CAB-O-SIL M 5® (fumed silica) which contains 4 to 24 weight ppm aluminum (apparently depending on the batch). Aerosil 200 fumed silica is another source of silica which contains <20 weight ppm aluminum. Other examples are tetraethyl orthosilicate which contains <0.2 weight ppm aluminum. Examples of materials which contain an excessive amount of aluminum (for purposes of this invention) are Ludox AS-40 (which typically contains 1000 weight ppm aluminum) and Nalco 2327 as used in U.S. Pat. No. 4,755,279, issued Jul. 5, 1988 to Unmuth et al., and U.S. Pat. No. 4,728,415, issued Mar. 1, 1988 to Unmuth et al. An analysis of the aluminum content of typical silica reagents is in Microporous and Mesoporous Materials, 32 (1999) 119-129. At high levels, the aluminum impurities in these reagents will impart excess strong acidity. Since aluminum tends to concentrate in the microporous weakly acidic molecular sieve product, the 500, 100 and 20 weight ppm limits for the silica reagent will translate into the following weight ppm aluminum limits for the microporous weakly acidic molecular sieve: less than 1000 weight ppm, preferably less than 200 weight ppm, and most preferably less than 20 weight ppm. U.S. Pat. No. 5,166,111, issued Nov. 24, 1992 to Zones et al., describes the preparation of a boron Beta microporous weakly acidic molecular sieve in accordance with this invention.

Forming the Catalyst Components

In order to be used in commercial applications, the catalyst components must be formed into a size suitable for use. The formed catalyst must have at least one dimension greater than 50 microns, preferably greater than 1/50″, most preferably greater than 1/20″. This forming can be done by techniques like pelletizing, extruding, and combinations thereof. Extrusion is the preferred method. Extruded materials can be cylinders, trilobes, fluted, or have other axially symmetrical shapes that promote diffusion and access to interior surfaces. To assist in forming a catalyst with good physical strength, binders are typically used.

Binders

In preparing catalysts for use in the present invention, the microporous weakly acidic molecular sieves are bound. They are preferably composited with matrix materials resistant to the temperatures and other conditions employed in hydrocarbon conversion processes. Also, it is preferred to use matrix materials that do not impart strong acidity into the catalyst. Such matrix materials can include active and inactive materials. Frequently, binders are added to improve the crush strength of the catalyst. The selection of binders and binding conditions depends on the microporous weakly acidic molecular sieve and its intended use.

The binder material can be selected from among the refractory oxides of metals of Groups 4A and 4B of the Periodic Table of the Elements. Particularly useful are the oxides of silicon, titanium and zirconium, with silica being preferred, especially low-aluminum silica. Combinations of such oxides with other oxides are also useful. For example, silica-magnesia, silica-zirconia, silica-thoria, silica-beryllia, silica-titania, titania-zirconia, and silica-magnesia-zirconia can be used, provided that these combinations do not form materials with strong acidity. (In contrast, the silica-alumina binder of aforementioned U.S. Pat. No. 4,728,415 used for borosilicate materials will impart strong acidity.) These oxides can be crystalline or amorphous, or can be in the form of gelatinous precipitates, colloids, sols, or gels. Silica in the form of a silica sol is a preferred binder. A preferred silica sol has about 30 wt % silica and contains small particles (7-9 nm in diameter), which result in catalysts with good attrition resistance and excellent crush strengths.

Forming pellets or extrudates from molecular sieves, including the microporous weakly acidic molecular sieves useful in this invention, generally involves using extrusion aids and viscosity modifiers in addition to the binders. These additives are typically organic compounds such as cellulose based materials, for example, Methocel® sold by Dow Chemical Co., ethylene glycol, and stearic acid. Many such compounds are known in the art. It is important that these additives do not leave a detrimental residue, i.e., one with undesirable reactivity or one that can block pores, after pelletizing. For this invention, it is especially desirable that such residues do not create strong acid functions in the catalyst. The above-described washing will remove low levels of these materials. The residue from the extrusion aid is preferably less than a few tenths of a percent, more preferably less than 0.1 wt %.

Methods for preparing catalyst compositions are well known to those skilled in the art and include such conventional techniques as spray drying, pelletizing, extrusion, various sphere-making techniques and the like. The newly developed method of in-extrudate formation of the zeolite/binder as described in U.S. Pat. No. 5,558,851, issued Sep. 24, 1996 to Miller and U.S. Pat. No. 5,514,362, issued May 7, 1996 to Miller, can also be used. The entire contents of these patents are incorporated herein by reference.

The relative proportions of molecular sieves to the binder/matrix can vary widely. Generally, the molecular sieve content ranges from between about 1 to about 99 weight percent, and more usually in the range of from about 5 to about 95 weight percent, of the dry composite, more typically 50-85%.

While laboratory use does not require it, the full-scale commercial use of the invention likely requires a bound microporous weakly acidic molecular sieve. It is preferred to use whole extrudates rather than crushed extrudates or unbound microporous weakly acidic molecular sieves in commercial operations. Bound microporous weakly acidic molecular sieves reduce the pressure drop through a reactor, provide improved flow rates, and are easier to load and unload.

Modifications

If the synthesized microporous weakly acidic molecular sieve or the bound microporous weakly acidic molecular sieve is found to have excess strong acidity, this function can be removed by modification methods. Suitable modification methods include acid extraction (typically with HCl), steaming, treatment with ammonium fluorosilicate and combinations thereof. All treatments involve contacting the microporous weakly acidic molecular sieve with aqueous solutions or steam at concentrations, temperatures, and times sufficient to remove the excess strong acidity, preferably without significant loss of the structure (as determined by XRD) or the pore volume.

Group 8 Metal

The catalyst comprises at least one Group 8 metal, preferably a noble metal (Pt, Pd, Rh, Ir, Ru, Os) and more preferably platinum. The content of the metal is preferably at or between 0.1 to 5 wt %, more preferably 0.1 to 3 wt %, and most preferably 0.3 to 1.5 wt %, based on the weight of the microporous weakly acidic molecular sieve. Platinum compounds that form positively charged platinum complex ions in solution are the preferred source of platinum. Platinum tetraamine chloride and nitrate are especially preferred.

To these Group 8 metals can also be added one or more non-Group 8 metals such as tin, indium and metals of Group 7B such as rhenium. Examples include Pt/Sn, Pt/Pd, Pt/Ni, and Pt/Re. These metals can be readily introduced into the composite employing a variety of known and conventional techniques, e.g., ion-exchange, incipient wetness, pore fill, impregnation, etc. Care should be taken so that the Group 8 metal, e.g., platinum, is incorporated in a manner that results in excellent and uniform dispersion. The incipient wetness impregnation method is preferred.

Reactor

The feed can be contacted with the catalyst in a fixed bed system, a moving bed system, a fluidized system, a batch system or combinations thereof. Reactors similar to those employed in hydrotreating and hydrocracking are suitable. Either a fixed bed system or a moving bed system is preferred. In a fixed bed system, the preheated feed is passed into at least one reactor that contains a fixed bed of the catalyst. The flow of the feed can be upward, downward or radial. The reaction is exothermic, and interstage cooling may be needed, especially if the content of heavy paraffins is high (>50 wt %) and the conversion is high (>50%). This cooling can be performed by injection of cool hydrogen between reactor beds. The reactors should be equipped with instrumentation to monitor and control temperatures, pressures and flow rates that are typically used in hydrocrackers.

Product Recovery and Subsequent Processing

The effluent from the hydroconversion reaction zone can be separated into the desired streams or fractions. Products such as solvents, jet fuel, jet fuel blend components, diesel fuel, diesel fuel blend components, feedstocks for linear alkyl benzene production, feedstock for lubricant base oil production, and by-product gases can be recovered using conventional techniques comprising distillation. A normal paraffin solvent can be recovered from products. If the n-paraffin content is not sufficient, it can be increased by use of well known adsorption techniques. One commercial adsorption process for removing branched hydrocarbons and aromatics from linear paraffins is known as the Molex or Sorbex process described in McPhee, Petroleum Technology Quarterly, pages 127-131, (Winter 1999/2000) which description is hereby incorporated by reference. This separation technology works by selectively adsorbing normal paraffins. However, the products from n-paraffin selective hydroconversion will be rich in normal paraffins and contain only small amounts of other species. Thus, it may be preferable to use an adsorbent that has a preference for adsorbing branched species over normal paraffins. Such materials have been described as inverse shape selective in Santilli D. S., Harris T. V., Zones S. I., Microporous Mater., 1329, 1993 hereby incorporated by reference. Other subsequent processing and integration options are described below.

Summary of Description of n-Paraffin Selective Hydroconversion Process

| Feature | Embodiment 1 | Embodiment 2 | Embodiment 3 | Embodiment 4 |
|---|---|---|---|---|
| Catalyst Performance in | | | | |
| n-C₁₆ hydroconversion at | | | | |
| 80% conversion. | | | | |
| Temperature, ° F. | ≦800 | ≦700 | ≦600 | ≦600 |
| Selectivity | | | | |
| C₆ i/n ratio | <0.75 | <0.2 | <0.05 | <0.01 |
| C₁₃ i/n ratio | | <2.0 | <0.5 | <0.1 |
| Catalyst Composition | | | | |
| Metal Choice | Group 8 | Pt, Pd, Rh, | Pt | Pt |
| | | Ir, Ru, Os | | |
| Metal, wt % | | 0.1-5 | 0.1-3 | 0.3-1.5 |
| Molecular Sieve Type | Microporous | Microporous Weakly Acidic | Borosilicate, | Borosilicate |
| | Weakly Acidic | Molecular Sieve | Titanosilicate | |
| | Molecular Sieve | | | |
| Aluminum, ppm | <1000 | <1000 | <200 | <20 |
| Molecular Sieve Pore | ≦12 rings | ≦12 rings | ≦10 rings | ≦10 rings |
| Types | | | | |
| Pore Dimensions | ≧1 | ≧1 | ≧2 | ≧3 |
| Molecular Sieve Structure | Weaklyacidic | SSZ-33, SSZ-46, SSZ-53, SSZ-55, | ZSM-5, ZSM-11, SSZ- | ZSM-5 |
| | molecular sieve | SSZ-57, SSZ-58, SSZ-59, SSZ-64, | 58, SSZ-57, SSZ-46, TS-1 | |
| | | ZSM-5, ZSM-11, TS-1, H-Y, SSZ-60, | | |
| | | SSZ-70 | | |
| Feed Properties | | | | |
| C₁₀₊ n-Paraffins, wt % | >5 | >50 | >80 | >80 |
| N, ppm | | <100 | <10 | <1 |
| S, ppm | | <100 | <100 | 0.1 to 10 |
| Oxygen, ppm | | | <10000 | <100 |
| Source | Petroleum or | Slack Wax, | Fischer Tropsch | Fischer Tropsch |
| | synthetic crude | Fischer Tropsch | | |
| Pretreatment | | Hydrotreated | Hydrotreated | Hydrotreated |
| Process Conditions | | | | |
| Temperature, ° F. | 600-850 | 700-800 | 725-775 | 725-775 |
| Pressure, psig | 50-5000 | 100-2000 | 250-1000 | 250-1000 |
| LHSV | 0.5-5 | 1-2 | 1-2 | 1-2 |
| Per-pass conv., % | 25-99 | 50-80 | 60-75 | 60-75 |

### EXAMPLE 1

### n-C₁₆ Test of Hydroconversion Catalysts

The hydroconversion of n-hexadecane is tested to identify catalysts which give a high selectivity to lighter normal paraffinic products over isomerized products. These results can be anticipated to be of value in selecting useful catalysts for n-paraffin hydroconversion of molecules of C₁₀ and greater, e.g., n-paraffin selective hydroconversion catalysts.

Unless otherwise noted in a specific example, the incorporation of noble metals (Pt or Pd) was carried out by ion-exchange at 212° F. for a minimum of 5 hours followed by filtration, washing, drying and calcination at 900° F.

The test conditions include a total pressure of 1200 psig, downflow hydrogen at 160 ml/min (when measured at 1 atmosphere pressure and 25° C.), downflow liquid feed rate of 1 ml/hr and the use of 0.5 g of catalyst loaded in the center of a 3 feet long by ¼ inch outside diameter stainless steel reactor tube (the catalyst is located centrally of the tube and extends about 1 to 2 inches in length) with alundum loaded upstream of the catalyst for preheating the feed. All materials were first reduced in flowing hydrogen at 570° F. overnight.

Products were analyzed by on-line capillary GC once every half hour. Raw data from the GC was collected by an automated data collection/processing system and conversions and selectivities calculated from the raw data. The catalyst was tested at 600° F. initially to determine the temperature range for the next set of measurements. The temperature was adjusted to give a conversion below 80%. Then the temperature was raised in 10° F. increments until the conversion exceeded 80%. Temperature was increased incrementally during a test and eight on-line samples (over four hours) were collected at each temperature. Conversions were defined as the conversion of n-C₁₆ to products with carbon numbers below n-C₁₆, thus the iso-C₁₆'s were not counted as a converted product. Yields were expressed as weight percent materials other than n-C₁₆ and included iso-C₁₆ as a product.

A catalyst, if it is to qualify as a catalyst of the invention, when tested in this manner, must convert at least 80% of the hexadecane to products having carbon numbers of 15 or less at temperatures of 800° F. or below, for example at temperatures of 700° F. or below, or at temperatures of 600° F. or below.

Also, when the catalyst is run at these conditions, but at temperatures which lead to 80% conversion of hexadecane to products having carbon numbers of 15 or less, the i/n ratio of the C₆ products will be less than 0.75, for example less than 0.2, less than 0.05, or less than 0.01.

Preferably the catalyst at 80% conversion as described above will also yield a C₁₃ i/n ratio of less than 2, for example less than 0.5, or less than 0.1.

Results are obtained at 80% conversion. But in cases where data is not obtained at precisely 80% conversion, results at 80% conversion can be obtained by linearly interpolating results from conversions slightly above and below 80%. The results at conversions above and below 80% conversion are obtained as close to 80% conversion as possible to assure that the linear interpolation is adequate. To determine that the linear interpolation is accurate, the temperatures and corresponding conversions should be selected so that the absolute value of percentage difference between the high and low values of the C₆ i/n ratio should not be greater than 40%.
Percentage Difference=Absolute value of {100*[(high C₆ i/n ratio−low C₆ i/n ratio)/low C₆ i/n ratio]}

In this calculation, if the low value of the C₆ i/n ratio is zero, the percentage difference is reported as zero, and the linear interpolation of the results is adequate.

This test can also be used to determine whether strong acid functions have been introduced into the catalyst during preparation. For example when bound and unbound materials are compared, a significant loss of activity or increase in the C₆ i/n ratio in the bound material is an indication of the introduction of acidity.

### EXAMPLE 2

### Preparation and Test of Pt/B-ZSM-5

ZSM-5 is a three-dimensional zeolite with all pores being composed of 10-ring structures. A sample of boron-ZSM-5 was made hydrothermally according to the following procedure. In a 23 cc Teflon liner, 2.5 gm of 25 wt % aqueous solution of tetrapropylammonium hydroxide, 1.25 gm 1N NaOH, 8 gm de-ionized water and 0.06 gm sodium borate decahydrate were thoroughly mixed until all sodium borate was dissolved. To this mixture, 0.9 gm CAB-O-SIL M-5 (fumed silica—98% SiO2) was added and the resulting gel was capped off and placed in a Parr autoclave and heated at 160° C. while rotating at about 43 rpm for 9 days. The resulting fine powder-liquid mixture was filtered and the obtained solid was thoroughly rinsed with water and air-dried overnight. The obtained solid was further dried in an oven at 120° C. for 3 hrs to yield 0.9 gm of Boron-ZSM-5 as told by X-ray analysis.

Pt/B-ZSM-5 was prepared according to the following procedure. A Boron-ZSM-5 sample, synthesized according to the example above, was calcined to remove the template. Calcination was done as follows. A thin bed of material is heated in a muffle furnace from room temperature to 120° C. at a rate of 1° C. per minute and held at 120° C. for 1 hour. The temperature is then ramped up to 540° C. at the same rate and held at this temperature for 5 hours, after which it is increased to 595° C. and held there for another 5 hours. A 50/50 mixture of air and nitrogen is passed over the sample at a rate of 20 standard cubic feet per minute during heating. The calcined sample was ion-exchanged with ammonium nitrate by heating the weakly acidic molecular sieve in water in the presence of ammonium nitrate (1 gm NH₄NO₃/1 gm weakly acidic molecular sieve in 10 gm water) for 3 hrs. The sample was then suspended in water (9 gm water/gm weakly acidic molecular sieve) and a solution of Pt(NH₃)₄(NO₃)₂ at a concentration which would provide 0.5 wt. % Pt with respect to the dry weight of the weakly acidic molecular sieve was added. The pH of the solution was adjusted to a pH of ˜9 with slow addition 0.15N ammonium hydroxide and stirred for 48 hours at 100° C. After cooling, the mixture was filtered through a glass frit, washed with de ionized water, and dried at 120° C. The sample was then calcined slowly up to 300° C. in air and held there for 3 hours. The boron content of the finished catalyst was 0.18 wt %

The catalyst was tested using n-C₁₆ as described in Example 1 with the results shown in Table 4. Values at 570° F. (57.9 percent conversion) and 580 (88% conversion) were used to linearly interpolate the results in the last column for 80% conversion. Since the C₆ i/n ratio was zero for both cases, the absolute value of percentage difference between these results was zero, and the linear interpolation of results was appropriate.

TABLE 4
Hydroconversion of n-C₁₆ over Pt/B-ZSM-5

| | | | | | | | | | | | Inter- |
|---|---|---|---|---|---|---|---|---|---|---|---|
| | Measured Data | | | | | | | | | | polated |
| Temp., ° F. | 510 | 520 | 541 | 545 | 559 | 570 | 580 | 590 | 600 | 610 | 577 |
| nC₁₆ Conv | 12.9 | 18.5 | 34.6 | 43.8 | 48.6 | 57.9 | 88 | 97.2 | 99.6 | 99.8 | 80 |
| i/n Ratios | | | | | | | | | | | |
| C₄ i/n | 0 | 0 | 0 | 0 | 0 | 0 | 0 | 0 | 0 | 0 | 0 |
| C₅ i/n | 0 | 0 | 0 | 0 | 0 | 0 | 0 | 0 | 0 | 0.01 | 0 |
| C₆ i/n | 0 | 0 | 0 | 0 | 0 | 0 | 0 | 0 | 0.01 | 0.01 | 0 |
| C₇ i/n | 0 | 0 | 0 | 0 | 0 | 0 | 0 | 0.01 | 0.01 | 0.01 | 0 |
| C₈ i/n | 0 | 0 | 0 | 0.01 | 0 | 0 | 0 | 0 | 0.01 | 0.01 | 0 |
| C₉ i/n | 0 | 0 | 0 | 0.02 | 0 | 0 | 0 | 0.01 | 0.01 | 0.02 | 0 |
| C₁₀ i/n | 0 | 0 | 0 | 0.01 | 0 | 0 | 0 | 0.01 | 0.01 | 0.03 | 0 |
| C₁₁ i/n | 0 | 0 | 0 | 0 | 0 | 0 | 0 | 0.01 | 0.02 | 0.07 | 0 |
| C₁₂ i/n | 0 | 0 | 0 | 0 | 0 | 0 | 0 | 0.01 | 0.03 | 0.02 | 0 |
| C₁₃ i/n | 0 | 0 | 0 | | 0 | 0 | 0 | 0.01 | 0.04 | 0.02 | 0 |
| Yields, % | | | | | | | | | | | |
| C₁ | 0.02 | 0 | 0.02 | 0.07 | 0.04 | 0.05 | 0.07 | 0.07 | 0.08 | 0.12 | 0.06 |
| C₂ | 0.02 | 0.13 | 0.16 | 0.28 | 0.13 | 0.19 | 0.28 | 0.26 | 0.32 | 0.50 | 0.26 |
| C₃ | 1.41 | 1.65 | 2.13 | 4.87 | 2.17 | 2.78 | 4.21 | 3.92 | 4.72 | 7.19 | 3.83 |
| C₄s | 2.59 | 2.99 | 3.82 | 8.60 | 3.89 | 4.95 | 7.52 | 7.10 | 8.73 | 13.27 | 6.84 |
| C₅s | 6.42 | 6.92 | 7.82 | 13.40 | 7.30 | 8.72 | 12.76 | 11.8 | 14.04 | 20.02 | 11.69 |
| C₆s | 15.81 | 15.53 | 14.63 | 17.64 | 11.79 | 12.31 | 16.06 | 13.58 | 14.37 | 18.04 | 15.06 |
| C₇-C₁₃ | 71.13 | 70.76 | 69.84 | 52.92 | 73.06 | 69.07 | 58.36 | 62.79 | 57.61 | 40.76 | 61.20 |
| iC₁₆ | 2.59 | 2.00 | 1.54 | 2.23 | 1.61 | 1.93 | 0.75 | 0.48 | 0.13 | 0.11 | 1.06 |
| Uniden. | 0 | 0 | 0.03 | 0 | 0.02 | 0 | 0 | 0 | 0 | 0 | 0 |
| Ratio C₃₊₄/C₇₋₁₃ | 0.06 | 0.07 | 0.09 | 0.25 | 0.08 | 0.11 | 0.20 | 0.18 | 0.23 | 0.50 | 0.18 |

The data in Table 4 also show that operation at high conversion tends to have undesirable features: higher catalyst temperatures, higher product i/n ratios, higher yields of light products, and lower yields of more valuable heavy products. This trend is increasingly evident when the conversion exceeds 99%. Thus, there is an incentive to operate at low conversions (25-50%), and recycle the unconverted normal paraffins. But the size of the process unit and catalyst charge will increase when operated at low conversion, and an economic optimum is expected for conversions between 25 and 99%, most preferably between 50 and 80%, very most preferably between 60 and 75%.

In comparison to other hydroconversion catalysts, Pt/B-ZSM-5 has several desirable features when comparisons are made at 80% conversion as shown in Table 6: lowest value of C₆ i/n ratio (zero at 80% conversion), lowest value of C₁₃ i/n ratio (zero at 80% conversion), lowest methane yields, and highest activity. Thus, it is the most preferred catalyst.

### EXAMPLE 3

### Preparation and Testing of Pt/B-SSZ-64

SSZ-64 is an unknown structure believed to be a multi-dimensional zeolite with pores being composed of 10 and/or 12-ring structures. Boron-SSZ-64 was made in a hydrothermal synthesis according to the procedure below. A 23 cc Teflon liner was charged with 4.8 gm of 0.62M aqueous solution of N-cyclobutylmethyl-N-ethylhexamethyleneiminium hydroxide (3 mmol SDA), 1.0 gm of 1M aqueous solution of NaOH (1 mmol NaOH) and 6.2 gm of de-ionized water. To this mixture, 0.06 gm of sodium borate decahydrate (0.157 mmol of Na₂B₄O₇.10H₂O; ˜0.315 mmol B₂O₃) was added and stirred until completely dissolved. Then, 0.9 gm of CAB-O-SIL M 5® (fumed silica) was added to the solution and the mixture was thoroughly stirred and the resulting gel was capped off and placed in a Parr autoclave and heated in an oven at 160° C. while rotating at 43 rpm for 12 days. The reaction mixture was filtered through a fritted-glass funnel and the collected solids were thoroughly washed with water and then rinsed with acetone (˜20 ml) to remove any organic residues. The solid was air-dried overnight and further dried in an oven at 120° C. for 1 hour to yield 0.85 gm of B-SSZ-64.

Pt/B-SSZ-64 was prepared according to the procedure described above for making Pt/B-ZSM-5 (Example 2) after a sample was calcined and ion-exchanged with NH₄NO₃. The pore volume of the calcined SSZ-64 was 0.19 cm³/g and SEM analysis shows 0.5 to 1.0μ sheet-like structures.

The catalyst was tested using n-C₁₆ as described in Example 1. Results were obtained at conversions near 80% and the linearly interpolated value at 80% conversion was derived. They are reported in Table 6.

### EXAMPLE 4

### Preparation and Testing of Pt/B-SSZ-58

SSZ-58 is a two-dimensional zeolite with all pores being composed of 10-ring structures. Boron-SSZ-58 was prepared in a hydrothermal synthesis using the same procedure described for the preparation of boron-SSZ-64 (Example 3) with the exception of using 1-cyclooctyl-1-butylpyrrolidinium hydroxide as the templating agent.

Pt/B-SSZ-58 was prepared according to the procedure described in Example 2 for making Pt/B-ZSM-5. The pore volume of the calcined SSZ-58 was 0.11 cm³/g and SEM analysis shows ˜2.5μ cylinders and rods. The Si/B molar ratio of the product was 42.

The catalyst was tested using n-C₁₆ as described in Example 1 results were obtained at conversions near 80% and the linearly interpolated value at 80% conversion was derived. They are reported in Table 6. This material showed the highest yields of the most valuable paraffins (C₇-C₁₃ range).

### EXAMPLE 5

### Preparation and Testing of Pt/B-SSZ-57

SSZ-57 is zeolite of unknown structure, but appears to be multidimensional and composed of 10 and/or 12 ring structures. Boron-SSZ-57 was prepared according the procedure described in Example 3 for making Boron-SSZ-64 using 1-cyclohexyl-1-butylpyrrolidinium hydroxide as the templating agent.

Pt/B-SSZ-57 was made according to procedure described in Example 2. The pore volume of the calcined SSZ-57 was 0.13 cm³/g and SEM analysis shows 0.3 to 0.5μ cubic structures.

The catalyst was tested using n-C₁₆ as described in Example 1. Results were obtained at conversions near 80% and the linearly interpolated value at 80% conversion was derived. They are reported in Table 6.

### EXAMPLE 6

### Preparation and Testing of Pd/SSZ-46 (Ti-ZSM-11)

SSZ-46 is a titanium-containing three-dimensional zeolite with the ZSM-11 structure with all pores being composed of 10-ring structures.

A sample of this material was made following Example 4 in U.S. Pat. No. 5,968,474, issued Oct. 19, 1999 to Nakagawa et al.

The catalyst was tested using n-C₁₆ as described in Example 1. Results were obtained only at one temperature which, while close to 80% conversion, was not exactly at this value. Nevertheless, this material shows the production of products rich in normal paraffins.

TABLE 5
Hydroconversion of n-C₁₆ over Pd/SSZ-46 (Ti-ZSM-11)

| | |
|---|---|
| Temp., ° F. | 733 |
| nC₁₆ Conv | 67.8 |
| i/n Ratios | |
| C₄ i/n | 0.30 |
| C₅ i/n | 0.18 |
| C₆ i/n | 0.17 |
| C₇ i/n | 0.14 |
| C₈ i/n | 0.16 |
| C₉ i/n | 0.14 |
| C₁₀ i/n | 0.13 |
| C₁₁ i/n | 0.14 |
| C₁₂ i/n | 0.18 |
| C₁₃ i/n | 0.21 |
| | 0.16 |
| Yields, % | |
| C_{1 +} C₂ | 0.51 |
| C₃ | 2.10 |
| C₄s | 4.84 |
| C₅s | 7.08 |
| C₆s | 7.75 |
| C₇-C₁₃ | 58.88 |
| iC₁₆ | 15.44 |
| Uniden. | 3.42 |
| Ratio C₃ + ₄/C₇-₁₃ | 0.12 |

In comparison to the results of Example 9 on Pt/B-ZSM-11, and Example 2 on Pt/B-ZSM-5, the sample of Pd/SSZ-46 gave higher amounts of isomerized lighter paraffin products and is significantly less active. Thus, boron is preferred over titanium, and both are preferred over aluminum which introduces undesirable strong acidity.

### EXAMPLE 7

### Preparation and Testing of Pt/B-SSZ-53

SSZ-53 is a one-dimensional zeolite with the pore being composed of 14-ring structures. Borosilicate SSZ-53 was synthesized according to the procedure described in Example 3 using trimethyl-(1-phenyl-cyclohexylmethyl)-ammonium hydroxide as the templating agent.

Pt/B-SSZ-53 was prepared according to the procedure detailed in Example 2. The pore volume of the calcined SSZ-53 was 0.13 cm³/g and SEM analysis shows 0.5 to 1.0μ lath-like structures. The Si/B molar ratio of the product was 39.

The catalyst was tested using n-C₁₆ as described in Example 1. Results were obtained at conversions near 80% and the linearly interpolated value at 80% conversion was derived. They are reported in Table 6.

### EXAMPLE 8

### Preparation and Testing of Pd/B-SSZ-58

A sample of B-SSZ-58 was made by the same procedure used in Example 5.

The purpose of this experiment was to determine the effect of the choice of the metal. Palladium/Boron-SSZ-58 was prepared according to the procedure described in Example 4 for making Pt/B-SSZ-58 by using Pd(NH₃)₄(NO₃)₂ solution in place of Pt(NH₃)₄(NO₃)₂ solution.

The catalyst was tested using n-C₁₆ as described in Example 1. Results were obtained at conversions near 80% and the linearly interpolated value at 80% conversion was derived. They are reported in Table 6. These results show that Pd can be used as a metal to give good selectivities to normal products, but it is not as effective as Pt. This catalyst did show the very desirable feature of giving the lowest yields of less desirable propane and butane.

### EXAMPLE 9

### Preparation and Testing of Pt/B-ZSM-11

Borosilicate ZSM-11 was synthesized according to the zeolite synthesis procedure described in Example 2 for making ZSM-5 using tetrabutylammonium hydroxide as the templating agent.

Pt/B-ZSM-11 was prepared according to Example 2.

The catalyst was tested using n-C₁₆ as described in Example 1. Results were obtained at conversions near 80% and the linearly interpolated value at 80% conversion was derived. They are reported in Table 6.

### EXAMPLE 10

### Preparation and Testing of Pt/B-SSZ-59

SSZ-59 is a one-dimensional zeolite with the pore being composed of 14-ring structures. Borosilicate SSZ-59 was synthesized according to the procedure described in Example 3 using 1-methy-1-(1-phenyl-cyclopentylmethyl)-piperidinium hydroxide as the templating agent.

Pt/B-SSZ-59 was made according to Example 2. The pore volume of the calcined SSZ-59 was 0.14 cm³/g and SEM analysis shows 0.5 along needles structures.

The catalyst was tested using n-C₁₆ as described in Example 1. Results were obtained at conversions near 80% and the linearly interpolated value at 80% conversion was derived. They are reported in Table 6.

### EXAMPLE 11

### Preparation and Testing of Pt/B-SSZ-55

SSZ-55 is a one-dimensional zeolite with the pore being composed of 12-ring structures. Borosilicate SSZ-55 was synthesized according to the weakly acidic molecular sieve synthetic procedure described in Example 3 using [1-(3-fluorophenyl)-cyclopentylmethyl]-trimethyl-ammonium hydroxide as the templating agent.

Pt/B-SSZ-55 was made according to Example 2. The pore volume of the calcined SSZ-55 was 0.15 cm³/g and SEM analysis shows 2 to 5μ rice grain-like structures.

The catalyst was tested using n-C₁₆ as described in Example 1. Results were obtained at conversions near 80% and the linearly interpolated value at 80% conversion was derived. They are reported in Table 6.

### COMPARATIVE EXAMPLE 12

### Preparation and Testing of Pd/H-Y

This is a comparative example of a commercial noble metal hydrocracking catalyst and is not the subject of this invention. The material was made following U.S. Pat. No. 5,141,909, issued Aug. 25, 1992 to Bezman, with the exception that the catalyst was not exposed to nitrogen. The aluminum in the Y zeolite of this sample imparts strong acidity which, while necessary for conventional hydrocracking, is not desirable in normal paraffin hydroconversion.

The catalyst was tested using n-C₁₆ as described in Example 1. Results were obtained at conversions near 80% and the linearly interpolated value at 80% conversion was derived. They are reported in Table 6. This demonstrates that this class of conventional hydrocracking catalysts gives mostly isomerized light paraffins and not normal paraffins as desired in the invention.

### COMPARATIVE EXAMPLE 13

### Preparation and Testing of Sulfided NiW/Silica Alumina

This is a comparative example of a commercial base metal hydrocracking catalyst and is not the subject of this invention. While it does not contain a zeolite, the aluminum in the amorphous silica alumina imparts strong acidity which, while necessary for conventional hydrocracking, is not desirable in normal paraffin hydroconversion.

The catalyst was tested using n-C₁₆ as described in Example 1. Results were obtained at conversions near 80% and the linearly interpolated value at 80% conversion was derived. They are reported in Table 6. This demonstrates that this class of conventional hydrocracking catalysts gives most isomerized light paraffins, high methane yield and not normal paraffins as desired in the invention. High methane yield indicates undesirable hydrogenolysis.

### EXAMPLE 14

### Preparation and Testing of Pt/B-SSZ-33

SSZ-33 is a multi-dimensional zeolite with the pore being composed of 12 and 10 ring structures. B-SSZ-33 was synthesized as follows: 2.0 Moles of trimethylammonium-8-tricyclo [5.2.1.0] decane in 3700 ml of water are mixed with 3600 ml of water, 92 grams of boric acid and 39 grams of solid NaOH. Once a clear solution is obtained, 558 grams of Cabosil M-5 are blended in and 5 grams of as-made B-SSZ-33 seed material are added. The entire contents have been mixed in the Hastelloy liner used in a 5-gallon autoclave (Autoclave Engineers). The reaction is stirred overnight at 200 rpm and at room temperature. Next, the reactor is ramped up to 160° C. over 12 hours and the stirring rate dropped to 75 rpm. The reaction is held under these conditions for 10 days of run time. The recovered, settled product is crystalline B-SSZ-33 in accord with U.S. Pat. No. 4,963,337, issued Oct. 16, 1990 to Zones. The water used in the present example should be distilled or deionized water in order to ensure that no aluminum is introduced. The other reactants and ingredients should also be selected to be essentially free of aluminum.

The calcined sample was impregnated by adding an aqueous ammonium nitrate solution (0.1506 gm Pt(NH₃)₄(NO₃)₂ in 35.3 gm deionized water) to 15.15 gm B-SSZ-33 at the dry weight at 350° C. After 48 hours at room temperature, the mixture was dried in a vacuum oven at 110° C. for 3 hours. Then the sample was calcined in air as follows: heat from room temperature to 120° C. in 1 hour, keep at 120° C. for 1 hour, heat from 120° C. to 300° C. in 3 hours, keep at 300° C. for 5 hours, then cooled down to room temperature, resulting in a calcined Pt/B-SSZ-33 catalyst containing 0.5 wt. % Pt on the dry zeolite. The Pt/B-SSZ-33 was then pelletized to 24-42 mesh for use of catalytic testing.

The catalyst was tested using n-C₁₆ as described in Example 1. Results were obtained at conversions near 80% and the linearly interpolated value at 80% conversion was derived. They are reported in Table 6.

TABLE 6
Comparison of Catalysts at 80% Hydroconversion of n-C₁₆ to Products Less than C₁₆

| | Example | | | | | |
|---|---|---|---|---|---|---|
| | 2 | 4 | 3 | 5 | 8 | 9 |
| Catalyst | PtBZSM5 | PtBSSZ58 | PtBSSZ64 | PtBSSZ57 | PdBSSZ58 | PtBZSM |
| | | | | | | 11 |
| Sieve | 3D 10R | 2D 10R | Unknown | Unknown | 2D 10R | 3D 10R |
| Structure | | | | | | |
| Micropore | | 0.11 | 0.19 | 0.13 | 0.11 | |
| vol. cm³/g | | | | | | |
| SEM Size, μ | | ˜2.5 | 0.5-1 | 0.3-0.5 | ˜2.5 | |
| Temp., ° F. | 577 | 695 | 655 | 691 | 694 | 605 |
| nC₁₆ Conv | 80.00 | 80.00 | 80.00 | 80.00 | 80.00 | 80.00 |
| i/n Ratios | | | | | | |
| C₄ i/n | 0.00 | 0.01 | 0.01 | 0.05 | 0.08 | 0.07 |
| C₅ i/n | 0.00 | 0.01 | 0.01 | 0.05 | 0.07 | 0.10 |
| C₆ i/n | 0.00 | 0.01 | 0.02 | 0.06 | 0.08 | 0.12 |
| C₇ i/n | 0.00 | 0.01 | 0.01 | 0.05 | 0.08 | 0.17 |
| C₈ i/n | 0.00 | 0.02 | 0.02 | 0.06 | 0.10 | 0.20 |
| C₉ i/n | 0.00 | 0.02 | 0.03 | 0.06 | 0.11 | 0.27 |
| C₁₀ i/n | 0.00 | 0.03 | 0.03 | 0.07 | 0.12 | 0.41 |
| C₁₁ i/n | 0.00 | 0.02 | 0.03 | 0.07 | 0.14 | 0.57 |
| C₁₂ i/n | 0.00 | 0.03 | 0.02 | 0.09 | 0.20 | 0.84 |
| C₁₃ i/n | 0.00 | 0.03 | 0.02 | 0.11 | 0.25 | 1.30 |
| C₄-C₁₃ i/n | 0.00 | 0.02 | 0.02 | 0.07 | 0.12 | 0.34 |
| Yields, wt % | | | | | | |
| C₁ | 0.06 | 0.19 | 0.89 | 0.26 | 0.16 | 0.32 |
| C₂ | 0.26 | 0.44 | 0.38 | 0.60 | 0.35 | 0.37 |
| C₃ | 3.83 | 2.69 | 3.52 | 3.26 | 1.79 | 2.81 |
| C₄s | 6.84 | 5.18 | 5.77 | 5.40 | 3.74 | 5.02 |
| C₅s | 11.69 | 7.70 | 7.97 | 6.99 | 6.25 | 6.32 |
| C₆s | 15.06 | 8.13 | 10.64 | 8.30 | 8.14 | 7.35 |
| C₇-C₁₃ | 61.21 | 72.15 | 65.40 | 66.54 | 66.80 | 60.32 |
| iC₁₆ | 1.06 | 3.52 | 5.40 | 8.65 | 12.75 | 17.41 |
| Unidentified | 0.00 | 0.00 | 0.02 | 0.00 | 0.00 | 0.10 |
| Ratio | 0.18 | 0.11 | 0.14 | 0.13 | 0.08 | 0.13 |
| C₃₊₄/C₇₋₁₃ | | | | | | |
| C₆ i/n low- | | | | | | |
| high | | | | | | |
| Delta | 0.00 | 0.00 | 0.00 | 0.01 | 0.00 | 0.04 |
| Percent | 0 | 0 | 0 | 17 | 0 | 40 |
| change | | | | | | |
| | Example | | | | | |
| | 7 | 14 | 10 | 11 | 12 | 13 |
| Catalyst | PtBSSZ53 | PtBSSZ33 | PtBSSZ59 | PtBSSZ55 | Pd/H-Y | NiMo |
| Sieve | 14R 1D | 12/10R 3D | 14R 1D | 12R 1D | 3D 12R | |
| Structure | | | | | | |
| Micropore | 0.13 | | 0.14 | 0.15 | | |
| vol. cm³/g | | | | | | |
| SEM Size, μ | 0.5-1 | | 0.5 | 2-5 | | |
| Temp., ° F. | 757 | 705 | 659 | 746 | 516 | 683 |
| nC₁₆ Conv | 80.00 | 80.00 | 80.00 | 80.00 | 80.00 | 80.00 |
| i/n Ratios | | | | | | |
| C₄ i/n | 0.21 | 0.13 | 0.37 | 0.33 | 2.40 | 0.60 |
| C₅ i/n | 0.20 | 0.18 | 0.51 | 0.45 | 3.71 | 0.81 |
| C₆ i/n | 0.20 | 0.21 | 0.51 | 0.48 | 4.59 | 0.89 |
| C₇ i/n | 0.18 | 0.26 | 2.95 | 0.60 | 5.23 | 1.12 |
| C₈ i/n | 0.18 | 0.32 | 0.66 | 0.75 | 6.17 | 1.57 |
| C₉ i/n | 0.19 | 0.35 | 0.74 | 0.87 | 6.80 | 1.98 |
| C₁₀ i/n | 0.21 | 0.38 | 4.14 | 1.18 | 8.61 | 2.70 |
| C₁₁ i/n | 0.26 | 0.44 | 1.24 | 1.55 | 7.85 | 3.29 |
| C₁₂ i/n | 0.29 | 0.51 | 1.53 | 1.97 | 9.59 | 3.99 |
| C₁₃ i/n | 0.28 | 0.60 | 1.61 | 2.52 | Infinite | 4.30 |
| C₄-C₁₃ i/n | 0.21 | 0.33 | 0.68 | 0.77 | 5.26 | 1.76 |
| Yields, wt % | | | | | | |
| C₁ | 0.99 | 0.35 | 0.71 | 2.12 | 0.00 | 6.81 |
| C₂ | 1.67 | 0.48 | 1.31 | 3.22 | 0.00 | 0.36 |
| C₃ | 5.28 | 2.40 | 4.79 | 6.84 | 1.44 | 1.74 |
| C₄s | 7.14 | 4.91 | 7.99 | 7.07 | 7.13 | 4.17 |
| C₅s | 7.75 | 6.86 | 9.12 | 7.30 | 10.38 | 5.47 |
| C₆s | 8.53 | 8.44 | 9.69 | 7.94 | 12.27 | 6.35 |
| C₇-C₁₃ | 58.40 | 64.10 | 50.87 | 48.17 | 54.34 | 55.45 |
| iC₁₆ | 10.23 | 12.45 | 15.51 | 17.34 | 14.45 | 19.65 |
| Unidentified | 0.00 | 0.11 | 0.00 | 0.00 | 0.00 | 0.00 |
| Ratio | 0.21 | 0.00 | 0.25 | 0.29 | 0.16 | 0.11 |
| C₃₊₄/C₇₋₁₃ | | | | | | |
| C₆ i/n low- | | | | | | |
| high | | | | | | |
| Delta | 0.03 | 0.02 | 0.12 | 0.17 | 1.37 | 0.17 |
| Percent | 16 | 20 | 29 | 40 | 26 | 29 |
| change | | | | | | |

### EXAMPLE 15

### Effect of Feedstock Sulfur

The Pt-B-ZSM-5 catalyst of Example 5 was sulfided at atmospheric pressure under the following conditions:
(1) First the 0.5 gram catalyst was purged in 100 cc/min N₂ at room temperature for 10 minutes. Then it was heated in N₂ at the same flow rate to 400° F. in 30 minutes and held at 400° F. in N₂ flow for 30 minutes.
(2) For the reduction of the catalyst, the catalyst was treated in a H₂ flow (100 cc/min) at 400° F. for 10 hours, then heated to 900° F. in the same H₂ flow in 1 hour and held at 900° F. in H₂ (100 cc/min) for 2 hours.
(3) Then the catalyst was cooled down to 800° F. in 30 minutes in H₂ flow (100 cc/min).
(4) The catalyst was then sulfided at 800° F. for 1 hour in a H₂ flow of 34 cc/min with an n-heptane feed containing 75 ppm sulfur (as dimethyl disulfide) at a feed rate of 38.4 cc/h.
(5) Then the n-heptane feed was stopped and the catalyst was heated from 800 to 900° F. in 30 minutes in a H₂ flow of 300 cc/min and kept at 900° F. in the same H₂ flow for 30 minutes.
(6) Finally, the catalyst was cooled down to room temperature in 100 cc/min H₂ in 2 hours.
(7) The catalyst was then ready for other catalytic test reactions.

This amount of sulfur corresponds to a S/Pt stoichiometry of 4.75 and is sufficient to produce detectable H₂S in the exit gas.

The sulfided catalyst was tested using n-C₁₆ as described in Example 1. Results were obtained at conversions near 80% and the linearly interpolated value at 80% conversion was derived. They are reported in Table 7 along with the results from the non-sulfided catalyst from Example 2.

TABLE 7
Comparison of
Non-sulfided, Sulfided, and Sulfided-plus-Titrated Pt-B-ZSM-5

| | Example | | | | |
|---|---|---|---|---|---|
| | | | Change | | Change |
| | 2 | 15 | 15 vs. 2 | 16 | 16 vs. 2 |
| Catalyst | PtBZSM5 | PtBZSM5 | | PtBZSM5 | |
| | No | Sulfided | | Sulfided, | |
| | heteroatoms | | | Titrated | |
| Temp., ° F. | 577 | 642 | +65 | 630 | +47 |
| nC₁₆ Conv | 80.00 | 80.00 | 0 | 80.00 | 0 |
| i/n Ratios | | | | | |
| C₄ i/n | 0.00 | 0.00 | 0.00 | 0.00 | 0.00 |
| C₅ i/n | 0.00 | 0.01 | +0.01 | 0.00 | 0.00 |
| C₆ i/n | 0.00 | 0.01 | +0.01 | 0.01 | +0.01 |
| C₇ i/n | 0.00 | 0.01 | +0.01 | 0.01 | +0.01 |
| C₈ i/n | 0.00 | 0.01 | +0.01 | 0.00 | 0.00 |
| C₉ i/n | 0.00 | 0.01 | +0.01 | 0.01 | +0.01 |
| C₁₀ i/n | 0.00 | 0.01 | +0.01 | 0.01 | +0.01 |
| C₁₁ i/n | 0.00 | 0.01 | +0.01 | 0.01 | +0.01 |
| C₁₂ i/n | 0.00 | 0.01 | +0.01 | 0.00 | 0.00 |
| C₁₃ i/n | 0.00 | 0.01 | +0.01 | 0.00 | 0.00 |
| C₄-C₁₃ i/n | 0.00 | 0.01 | +0.01 | 0.01 | +0.01 |
| Yields, wt % | | | | | |
| C₁ | 0.06 | 0.00 | −0.06 | 0.00 | −0.06 |
| C₂ | 0.26 | 0.41 | +0.15 | 0.39 | +0.13 |
| C₃ | 3.83 | 3.07 | −0.76 | 3.18 | −0.65 |
| C₄s | 6.84 | 5.43 | −1.14 | 5.72 | −1.12 |
| C₅s | 11.69 | 7.87 | −3.82 | 8.50 | −3.19 |
| C₆s | 15.06 | 9.60 | −5.46 | 10.34 | −4.72 |
| C₇-C₁₃ | 61.21 | 71.53 | +10.32 | 68.96 | +7.75 |
| iC₁₆ | 1.06 | 2.10 | +1.40 | 2.79 | +1.73 |
| Unidentified | 0.00 | 0.00 | 0 | 0.13 | +0.13 |
| Ratio C₃₊₄/ | 0.18 | 0.12 | −0.06 | 0.19 | +0.01 |
| C₇₋₁₃ | | | | | |
| C₆ i/n low-high | | | | | |
| Delta | 0.00 | 0.00 | | 0 | |
| Percent change | 0 | 0 | | 0 | |

These results show that exposing the catalyst to sulfur results in some activity loss, and a slight increase in product i/n ratio, but give a beneficial increase in the production of more valuable heavy normal paraffins. This shift in yields reduces the consumption of valuable hydrogen by about 100 SCF/B. Thus, the sulfur content of the feed should be <100 ppm, for example between 0.1 and 10 ppm. Sulfur content can be regulated by hydrotreating high sulfur feeds, or increasing the sulfur content of low sulfur feeds (such as a Fischer Tropsch-derived feed). The sulfur content of a low sulfur feed can be increased by blending it with a high sulfur feed (such as a petroleum feed) or by pre-sulfiding the catalyst, or by continuously adding a sulfur containing component (dimethyl disulfide, mercaptans, hydrogen sulfide, extracts from a sweetening operation, and the like.) If sulfur is needed to improve selectivity, it can be added continuously or intermittently.

### EXAMPLE 16

### Effect of Feedstock Nitrogen

The Pt-B-ZSM-5 catalyst of Example 15 was further tested with an n-C₁₆ feed containing 5 ppm by weight nitrogen as butyl amine. Under the reaction conditions, the butyl amine decomposes giving ammonia and butene. The ammonia serves to titrate strong Brönsted acid sites. With this feed the catalyst was tested at temperatures from 550 to 670° F., by increasing temperature in 110° F. increments as described in Example 1. Overall n-C₁₆ conversion increased from 9.9 to 99.7% over this temperature range. Results were obtained at conversions near 80% and the linearly interpolated value at 80% conversion was derived. They are reported in Table 7 along with the results from the non-sulfided catalyst from Example 2.

These results show that the non-acidic borosilicate is remarkably tolerant of nitrogen. Activity improves upon exposure to nitrogen. Without wishing to be bound by any theory, it is believed this is attributed to a stripping of some sulfur from the catalyst and a slight reversal of the change seen between Experiment 15 on the sulfided catalyst and Experiment 2 on the catalyst prior to exposure to either heteroatom. Nitrogen is believed to have little or not effect on the catalyst.

### EXAMPLE 17

### Effect of Boron Content in B-ZSM-5

A series of Pt-B-ZSM-5 catalysts were prepared without aluminum and with varying levels of boron.

### COMPARATIVE EXAMPLE 17a

### Synthesis of the All-Silica ZSM-5(sample 53353)

This sample of all-silica-ZSM-5 was made hydrothermally according to the following procedure. In a 23 cc Teflon liner, 2.5 gm of 25 wt % aqueous solution of tetrapropylammonium hydroxide, 1.25 gm 1N NaOH, 8 gm de-ionized water and I gm CAB-O-SIL M-5 (fumed silica—98% SiO₂) were thoroughly mixed. The resulting gel was capped off and placed in a Parr autoclave and heated at 160° C. while rotating at about 43 rpm for 12 days. The resulting fine powder-liquid mixture was filtered and the obtained solid was thoroughly rinsed with water and air-dried overnight. The obtained solid was further dried in an oven at 120° C. for 3 hrs to yield 0.88 gm of all-silica-ZSM-5(X-ray analysis).

Pt/All-sillica-ZSM-5 was prepared according to the following procedure. The all-silica-ZSM-5 sample, synthesized according to the example above, was calcined to remove the template. Calcination was done as follows. A thin bed of material is heated in a muffle furnace from room temperature to 120° C. at a rate of 1° C. per minute and held at 120° C. for 1 hour. The temperature is then ramped up to 540° C. at the same rate and held at this temperature for 5 hours, after which it is increased to 595° C. and held there for another 5 hours. A 50/50 mixture of air and nitrogen is passed over the sample at a rate of 20 standard cubic feet per minute during heating. The calcined sample was ion-exchanged with ammonium nitrate by heating the zeolite in water in the presence of ammonium nitrate (1 gm NH₄NO₃/1 gm zeolite in 10 gm water) for 3 hours. The sample was then suspended in water (9 gm water/gm zeolite) and a Pt(NH₃)₄(NO₃)₂ solution at a concentration which would provide 0.5 wt. % Pt with respect to the dry weight of the zeolite was added. The pH of the solution was adjusted to a pH of 9 with slow addition 0.15 N ammonium hydroxide and stirred for 48 hours at 100° C. After cooling, the mixture was filtered through a glass frit, washed with de ionized water, and dried at 120° C. The sample was then calcined slowly up to 300° C. in air and held there for 3 hours.

A check of the platinum content of this sample showed that little had been incorporated. In contrast, samples with boron>0.10 wt % aluminum, or combinations incorporated the expected amount.

17b. Synthesis of the B-ZSM-5

This sample was synthesized according to the synthesis procedure described above for the synthesis of the all-silica sample with exception of adding 0.01 gm of Na₂B₄O₇(anhydrous). The Pt-version was synthesized according the above procedure. Platinum was found in this sample, but at less than the amount expected. This is an indication that the silanol groups in the all-silica sample are sufficiently strong to act as effective exchange sites for the metal.

Analysis of these zeolites and their catalytic performance is shown in the Table 8.

TABLE 8
Effect of Boron Level in Pt-B-ZSM-5 catalysts

| | Example | | |
|---|---|---|---|
| | Comp. 17a | 17b | 2 |
| Boron Content, wt % | <0.007 | 0.10 | 0.18 |
| Aluminum Content, ppm | <20 | <20 | <20 |
| Temp., ° F. | 750 | 753 | 577 |
| nC₁₆ Conv | 10.8 | 80.00 | 80.00 |
| i/n Ratios | | | |
| C₄ i/n | 0.43 | 0.50 | 0.00 |
| C₅ i/n | 0.43 | 0.49 | 0.00 |
| C₆ i/n | 0.43 | 0.50 | 0.00 |
| C₇ i/n | 0.37 | 0.54 | 0.00 |
| C₈ i/n | 0.41 | 0.55 | 0.00 |
| C₉ i/n | 0.19 | 0.58 | 0.00 |
| C₁₀ i/n | 0.47 | 0.76 | 0.00 |
| C₁₁ i/n | 0.44 | 1.03 | 0.00 |
| C₁₂ i/n | 0.11 | 1.04 | 0.00 |
| C₁₃ i/n | 0.16 | 0.89 | 0.00 |
| C₄-C₁₃ i/n | 0.36 | 0.60 | 0.00 |
| Yields, wt % | | | |
| C₁ | 0.02 | 0.10 | 0.06 |
| C₂ | 0.17 | 0.13 | 0.26 |
| C₃ | 3.29 | 4.45 | 3.83 |
| C₄s | 6.19 | 9.01 | 6.84 |
| C₅s | 7.51 | 11.30 | 11.69 |
| C₆s | 7.99 | 12.20 | 15.06 |
| C₇-C₁₃ | 38.31 | 45.63 | 61.21 |
| iC₁₆ | 36.53 | 17.18 | 1.06 |
| Unidentified | 0 | 0.00 | 0.00 |
| Ratio C₃ + ₄/C₇-₁₃ | | 0.30 | 0.18 |
| C₆ i/n low-high | | | |
| Delta | | 0.02 | 0.00 |
| Percent change | | 4.17 | 0 |

The all-silica sample (17a) was inactive with conversion being only 10.8% at 750° F. Results at 80% conversion could not be determined experimentally and were estimated to require temperatures in excess of 970° F. It contains an insufficient amount of boron for use. The sample with 0.1 wt % boron was more active, but not as active as the sample with 0.18 wt % boron. These results demonstrate that for the ZSM-5 structure, the boron content should be greater than about 0.05 wt % (equivalent to 800° F. catalyst temperature or better), for example greater than about 0.125 wt % (equivalent to 700° F. catalyst temperature or better), or greater than about 0.15 wt % (equivalent to 600° F. catalyst temperature or better). These relationships between boron content and catalyst activity are for ZSM-5. They likely depend on the structure of the weakly acidic molecular sieve.

### EXAMPLE 18

### Effect of Aluminum Content in B-ZSM-5

A series of Pt-B-ZSM-5 catalysts were prepared with varying trace levels of aluminum.

18a. Synthesis of the Aluminum-Containing B-ZSM-5

This sample of a trace aluminum-containing boron-ZSM-5 was made hydrothermally according to the following procedure. In a 23 cc Teflon liner, 0.8 gm of 40 wt % aqueous solution of tetrapropyl-ammonium hydroxide, 1.25 gm 1N NaOH, 10 gm de-ionized water and 0.033 gm of sodium borate were mixed until all sodium borate were dissolved. Then, 0.9 gm CAB-O-SIL M-5 (fumed silica—98% SiO₂) and 0.001 gm of Na-Y zeolite (SiO₂/Al₂O₃=5: 0.00011.2 gm Al which amounts to ˜115 ppm of the solids or ˜10 ppm of the entire gel mixture) were added, and the mixture was thoroughly mixed. The resulting gel was capped off and placed in a Parr autoclave and heated at 160° C. while rotating at about 43 rpm for 12 days. The resulting fine powder-liquid mixture was filtered and the obtained solid was thoroughly rinsed with water and air-dried overnight. Then, the dried product was suspended in 5 ml of 0.01N HCl and heated (static) at 80° C. to ensure the complete destruction of any unreacted Na-Y. The solid was filtered and dried in open air and further dried in an oven at 120° C. for 3 hrs to yield 0.83 gm of ZSM-5 (X-ray analysis).

The calculated aluminum content of this material was 145 ppm, and the measured boron content was 0.21 wt %.

Pt/ZSM-5 of this sample was prepared according to the following procedure. The sample, synthesized according to the example above, was calcined to remove the template. Calcination was done as follows. A thin bed of material is heated in a muffle furnace from room temperature to 120° C. at a rate of 1° C. per minute and held at 120° C. for 1 hour. The temperature is then ramped up to 540° C. at the same rate and held at this temperature for 5 hours, after which it is increased to 595° C. and held there for another 5 hours. A 50/50 mixture of air and nitrogen is passed over the sample at a rate of 20 standard cubic feet per minute during heating. The calcined sample was ion-exchanged with ammonium nitrate by heating the zeolite in water in the presence of ammonium nitrate (1 gm NH₄NO₃/1 gm zeolite in 10 gm water) for 3 hrs. The NH₄-form of this sample was then suspended in water (9 gm water/gm zeolite) and a Pt(NH₃)₄(NO₃)₂ solution at a concentration which would provide 0.5 wt. % Pt with respect to the dry weight of the zeolite was added. The pH of the solution was adjusted to a pH of ˜9.2 with slow addition 0.15N ammonium hydroxide and stirred for 48 hours at 95° C. After cooling, the mixture was filtered through a glass frit, washed with de-ionized water, and dried at 120° C. The sample was then calcined slowly up to 300° C. in air and held there for 3 hours. The sample was then pelletized on a Carver Press, transformed to a 24-40 mesh and used as the catalyst.

18b. Synthesis of the Aluminum-Containing B-ZSM-5

This sample of aluminum-containing boron-ZSM-5 was made hydrothermally according to the following procedure. In a 23 cc Teflon liner, 2.45 gm of 25 wt % aqueous solution of tetrapropylammonium hydroxide, 1.2 gm 1N NaOH, 8.35 gm deionized water and 0.06 gm of sodium borate decahydrate were mixed until all sodium borate were dissolved. Then, 0.9 gm CAB-O-SIL M-5 (fumed silica—98% SiO2) and 0.0025 gm of Na-A zeolite (SiO₂/Al₂O₃=1.7; 00037 gm Al which amounts to ˜385 ppm) were added, and the mixture was thoroughly mixed. The resulting gel was capped off and placed in a Parr autoclave and heated at 160° C. while rotating at about 43 rpm for 12 days. The resulting fine powder-liquid mixture was filtered and the obtained solid was thoroughly rinsed with water and air-dried overnight. Then, the dried product was suspended in 5 ml of 0.01N HCl and heated (static) at 80° C. to ensure the complete destruction of any un-reacted Na-Y. The solid was filtered and dried in open air and further dried in an oven at 120° C. for 3 hours to yield 0.8 gm of ZSM-5 (X-ray analysis).

The calculated aluminum content of this material was 402 ppm, and the measured boron content was 0.21 wt %.

Pt/Al-B-ZSM-5 of this sample was prepared according to the procedure used for sample 18a.

18c. Synthesis of the Aluminum-Containing B-ZSM-5

This sample was synthesized according to the procedure for sample 18b but using 0.005 gm N-A zeolite (0.00074 gm Al).

The calculated aluminum content of this material was 785 ppm, and the measured boron content was 0.22 wt %.

Pt/Al-B-ZSM-5 of this sample was prepared according to the procedure used for sample 18a.

18d. Synthesis of the Aluminum-Containing B-ZSM-5

This sample was synthesized according to the procedure above but using 0.007 gm N-A zeolite (0.001 gm Al).

The calculated aluminum content of this material was 1000 ppm, and the measured boron content was 0.18 wt %. Pt/Al-B-ZSM-5 of this sample was prepared according to the procedure used for sample 18a.

18e. Synthesis of the Aluminum-Containing B-ZSM-5

This sample was synthesized according to the procedure above but using 0.01 gm N-A zeolite (0.00148 gm Al).

The calculated aluminum content of this material was 1500 ppm, and the measured boron content was 0.17 wt %.

Pt/Al-B-ZSM-5 of this sample was prepared according to the procedure used for sample 18a.

Analysis of these zeolites and their catalytic performance is shown in the Table 9.

TABLE 9
Effect of Trace Aluminum Level in Pt-B-ZSM-5 catalysts

| | Example | | | | | |
|---|---|---|---|---|---|---|
| | 2 | 18a | 18b | 18c | 18d | 18e |
| Measured | 0.18 | 0.21 | 0.21 | 0.22 | 0.18 | 0.17 |
| Boron | | | | | | |
| Content, wt % | | | | | | |
| Calculated | <20 | 145 | 402 | 785 | 1000 | 1500 |
| Aluminum | | | | | | |
| Content, ppm | | | | | | |
| Temp., ° F. | 577 | 567 | 559 | 593 | 567 | 563 |
| nC₁₆ Conv | 80.00 | 80.00 | 80.00 | 80.00 | 80.00 | 80.00 |
| i/n Ratios | | | | | | |
| C₄ i/n | 0.00 | 0.02 | 0.26 | 0.25 | 0.22 | 0.22 |
| C₅ i/n | 0.00 | 0.03 | 0.29 | 0.28 | 0.24 | 0.24 |
| C₆ i/n | 0.00 | 0.03 | 0.34 | 0.35 | 0.29 | 0.29 |
| C₇ i/n | 0.00 | 0.05 | 0.42 | 0.45 | 0.38 | 0.37 |
| C₈ i/n | 0.00 | 0.04 | 0.40 | 0.44 | 0.37 | 0.35 |
| C₉ i/n | 0.00 | 0.04 | 0.42 | 0.48 | 0.40 | 0.38 |
| C₁₀ i/n | 0.00 | 0.06 | 0.51 | 0.74 | 0.61 | 0.54 |
| C₁₁ i/n | 0.00 | 0.06 | 0.62 | 1.04 | 0.82 | 0.74 |
| C₁₂ i/n | 0.00 | 0.07 | 0.71 | 0.57 | 1.00 | 0.93 |
| C₁₃ i/n | 0.00 | 0.03 | 0.77 | 0.90 | 1.17 | 1.20 |
| C₄-C₁₃ i/n | 0.00 | 0.04 | 0.40 | 0.44 | 0.39 | 0.40 |
| Yields, wt % | | | | | | |
| C₁ | 0.06 | 0.06 | 0.00 | 0.03 | 0.00 | 0.00 |
| C₂ | 0.26 | 0.23 | 0.15 | 0.07 | 0.11 | 0.11 |
| C₃ | 3.83 | 3.29 | 7.32 | 4.98 | 5.65 | 4.97 |
| C₄s | 6.84 | 6.04 | 11.52 | 8.89 | 10.20 | 8.87 |
| C₅s | 11.69 | 9.56 | 13.30 | 11.09 | 12.65 | 10.95 |
| C₆s | 15.06 | 11.65 | 13.54 | 11.88 | 13.32 | 11.75 |
| C₇-C₁₃ | 61.21 | 65.12 | 47.72 | 47.09 | 47.72 | 51.51 |
| iC₁₆ | 1.06 | 4.04 | 6.47 | 15.97 | 10.34 | 11.84 |
| Unidentified | 0.00 | 0.00 | 0.00 | 0.00 | 0.00 | 0.00 |
| Ratio C₃₊₄/ | 0.18 | 0.14 | 0.39 | 0.29 | 0.33 | 0.27 |
| C₇₋₁₃ | | | | | | |
| C₆ i/n low-high | | | | | | |
| Delta | 0.00 | 0.01 | 0.04 | 0.04 | 0.04 | 0.03 |
| Percent change | 0 | 33.3 | 13.3 | 11.76 | 7.14 | 10.71 |

These results demonstrate that for the production of a lighter product with a low i/n paraffin ratio from a n-paraffin the aluminum content of the molecular sieve should be less than 1000 ppm, for example less than 200 ppm or less than 20 ppm. Because the molecular sieve structure may also influence the product i/n ratio, the exact aluminum content needed for a given structure to obtain the desired product i/n ratio may be less than these values.

### EXAMPLE 19

### Tests with Fischer Tropsch Wax

A 10 gram sample of Pt/B-SSZ-58 was prepared following the procedure of Example 4. It was tested in a trickle bed microunit operating in downflow mode. The catalyst was converted to pellets and 7.0 cc was placed in the reactor. It was reduced in hydrogen at 450° F. The reactor inlet pressure was adjusted to the target (1000 psig initially) and the gas rate adjusted to 10,000 SCFB. The feed was then started at 1.0 LHSV. Catalyst temperature was adjusted to give a range of feed conversions.

The microunit configuration permitted calculation of product yields, feed conversions and mass balances based on detailed feed and product analysis (ASTM D-2887 simulated distillation, gas analysis).

The feedstock was a Fischer Tropsch wax prepared from a slurry bed process using a cobalt catalyst. The wax was hydrotreated to remove impurities (olefins, nitrogen, oxygenates, solids, etc). The properties of the hydrotreated wax are shown in Tables 10 and 11.

This catalyst was evaluated at a variety of pressures and temperatures with yields shown in Table 12. When the pressure was reduced, the catalyst's activity increased.

TABLE 10
Feed Analysis

Feed for Example
No.

| | 19, 20 | 21 |
|---|---|---|
| | HDT FT Wax | Daqing 650N |
| API Gravity | 41.1 | 32.0 |
| Sulfur, weight ppm | <1 | <6 |
| Nitrogen, weight | <1 | 1.2 |
| ppm | | |
| Oxygen, wt % | 0.05 | 0 |
| Carbon, wt % | 84.91 | 85.87 |
| Hydrogen, wt % | 15.04 | 14.13 |
| MW | 444 | 530 |
| Wax, wt % | 100.0 | 53.0 |
| Oil-in-wax, wt % | 0.0 | 12.4 |
| D2887 Distil., ° F. | | |
| by wt % | | |
| 0.5/5 | 512/598 | 678/796 |
| 10/30 | 642/732 | 850/943 |
| 50/ | 817 | 979 |
| 70/90 | 908/1035 | 1001/1036 |
| 95/99 | 1096/1199 | 1050/1071 |

TABLE 11
Normal Paraffin Analyses of Feedstocks

| | |
|---|---|
| F-T-Wax | Daqing 650N |

| | | | | |
|---|---|---|---|---|
| Paraffin Analysis, wt % | n-P | Non-n P | n-P | Non-n P |

| | | | |
|---|---|---|---|
| C12− | 3.22 (mostly n-P) | 0.03 | 0.01 |

| | | | | |
|---|---|---|---|---|
| C12 | 0.05 | 0.00 | 0.03 | 0.06 |
| C13 | 0.18 | 0.01 | 0.05 | 0.09 |
| C14 | 0.45 | 0.03 | 0.05 | 0.07 |
| C15 | 0.85 | 0.05 | 0.07 | 0.07 |
| C16 | 1.33 | 0.07 | 0.07 | 0.09 |
| C17 | 1.89 | 0.10 | 0.08 | 0.03 |
| C18 | 2.52 | 0.14 | 0.08 | 0.06 |
| C19 | 3.17 | 0.20 | 0.10 | 0.05 |
| C20 | 3.79 | 0.24 | 0.11 | 0.03 |
| C21 | 4.31 | 0.26 | 0.14 | 0.04 |
| C22 | 4.71 | 0.30 | 0.21 | 0.08 |
| C23 | 4.93 | 0.32 | 0.37 | 0.11 |
| C24 | 4.97 | 0.33 | 0.48 | 0.28 |
| C25 | 4.88 | 0.33 | 0.69 | 0.32 |
| C26 | 4.70 | 0.33 | 0.63 | 0.33 |
| C27 | 4.45 | 0.34 | 0.75 | 0.50 |
| C28 | 4.19 | 0.35 | 0.70 | 0.48 |
| C29 | 3.91 | 0.33 | 0.71 | 0.50 |
| C30 | 3.62 | 0.31 | 0.72 | 0.44 |
| C31 | 3.35 | 0.32 | 0.72 | 0.54 |
| C32 | 3.20 | 0.32 | 0.81 | 0.58 |
| C33 | 2.84 | 0.30 | 0.81 | 0.67 |
| C34 | 2.64 | 0.25 | 0.96 | 0.63 |
| C35 | 2.41 | 0.24 | 0.89 | 0.79 |
| C36 | 2.50 | 0.23 | 1.33 | 1.89 |
| C37 | 1.98 | 0.22 | 1.66 | 3.98 |
| C38 | 1.78 | 0.19 | 1.90 | 6.49 |
| C39 | 1.58 | 0.17 | 2.13 | 8.82 |
| C40 | 2.33 | 0.19 | 2.18 | 9.25 |
| C41 | 1.23 | 0.23 | 1.50 | 8.80 |
| C42 | 1.06 | 0.14 | 1.02 | 6.70 |
| C43 | 0.84 | 0.13 | 0.82 | 5.59 |
| C44 | 2.11 | 0.20 | 1.54 | 5.43 |
| C45 | 0.33 | 0.15 | 0.49 | 4.31 |
| C46 | 0.15 | 0.05 | 0.45 | 2.53 |
| C47 | 0.06 | 0.05 | 0.51 | 1.34 |
| C48 | 0.02 | 0.00 | 0.43 | 0.59 |
| C49 | 0.00 | 0.01 | 0.27 | 0.21 |
| C50+ | 0.00 | 0.00 | 0.20 | 0.54 |
| Sum | 89.33 | 10.68 | 26.69 | 73.31 |

TABLE 12
Hydroconversion of Fischer Tropsch Wax With Pt/B-SSZ-58

| | Run Hours | | | | |
|---|---|---|---|---|---|
| | 402 | 666 | 858 | 906 | 1074 |
| Temp., ° F. | 702 | 731 | 722 | 715 | 715 |
| WHSV | 1.19 | 1.19 | 1.19 | 1.20 | 1.19 |
| Tot. P, psig | 1113 | 1107 | 1104 | 1099 | 259 |
| Gas Rate, | 9755 | 9740 | 9749 | 9693 | 9725 |
| SCFB | | | | | |
| Conv <650° F., | ˜10 | 92.99 | 93.8 | 74.27 | 80.46 |
| Wt % | | | | | |
| Yields, | | | | | |
| Wt % | | | | | |
| Methane | | 0.17 | 0.14 | 0.10 | 0.20 |
| Ethane | | 0.29 | 0.23 | 0.16 | 0.32 |
| Propane | | 1.74 | 1.45 | 0.88 | 1.42 |
| i-Butane | | 0.05 | 0.04 | 0.02 | 0.03 |
| n-Butane | | 3.38 | 3.05 | 1.78 | 2.29 |
| C₅-180° F. | | 10.41 | 9.90 | 6.09 | 9.11 |
| 180-250° F. | | 3.68 | 3.45 | 2.16 | 2.33 |
| 250-550° F. | | 62.93 | 61.18 | 46.35 | 50.16 |
| 550-700° F. | | 17.46 | 22.09 | 34.31 | 29.23 |
| 700° F.+ | | 2.83 | 1.48 | 10.70 | 7.60 |
| D2887 | | | | | |
| Distil., ° F. | | | | | |
| by wt % | | | | | |
| St/5% | 118/411 | 91/207 | 33/209 | 91/256 | 90/256 |
| 10/30% | 478/621 | 257/383 | 259/385 | 302/454 | 301/420 |
| 50% | 707 | 454/ | 457 | 549 | 521 |
| 70/90% | 786/921 | 522/623 | 548/625 | 625/713 | 600/694 |
| 95/99% | 991/1097 | 670/771 | 658/713 | 743/805 | 732/788 |

A GC analysis of the converted portions of the whole liquid products from the unit showed large peaks due to normal paraffins with only small intermediate peaks. Portions of the products from hours 666 were distilled by D-1160 at a 650° F. cut point to produce a bottoms product, overhead product, and light material which was in the trap. The products were analyzed for density, normal paraffin content and distribution, cetane index, as shown in Table 13.

TABLE 13
Analysis of the Products from the Fischer Tropsch Feedstock

| | Trap | | Overhead | | Bottoms | |
|---|---|---|---|---|---|---|
| API Gravity | 68.9 | | 53.8 | | 42.4 | |
| Volume, % | 20.5 | | 69.9 | | 9.6 | |
| Weight, % | 19.1 | | 70.6 | | 10.3 | |
| Cetane Index | | | 78 | | | |
| n-Paraffin | | | | | | |
| Analysis, wt % | n-P | Non n-P | n-P | Non n-P | n-P | Non n-P |
| C4 | 0.24 | 0.00 | 0.00 | 0.00 | 0.00 | 0.00 |
| C5 | 2.10 | 0.14 | 0.00 | 0.00 | 0.00 | 0.00 |
| C6 | 6.82 | 4.11 | 0.00 | 2.44 | 0.00 | 0.00 |
| C7 | 15.91 | 0.43 | 0.01 | 0.03 | 0.01 | 0.00 |
| C8 | 25.09 | 0.99 | 0.34 | 0.04 | 0.02 | 0.01 |
| C9 | 20.90 | 1.22 | 2.27 | 0.08 | 0.03 | 0.06 |
| C10 | 10.43 | 0.99 | 5.66 | 0.21 | 0.04 | 0.07 |
| C11 | 3.93 | 0.89 | 9.22 | 0.39 | 0.01 | 0.10 |
| C12 | 1.38 | 1.18 | 10.99 | 0.50 | 0.01 | 0.07 |
| C13 | 0.47 | 1.06 | 10.52 | 0.57 | 0.00 | 0.05 |
| C14 | 0.20 | 0.51 | 10.65 | 0.61 | 0.01 | 0.06 |
| C15 | 0.11 | 0.18 | 10.31 | 0.69 | 0.00 | 0.03 |
| C16 | 0.07 | 0.14 | 9.02 | 0.77 | 0.02 | 0.03 |
| C17 | 0.04 | 0.10 | 7.50 | 0.80 | 0.37 | 0.06 |
| C18 | 0.03 | 0.09 | 5.80 | 0.78 | 2.56 | 0.19 |
| C19 | 0.00 | 0.14 | 3.79 | 0.62 | 6.90 | 0.62 |
| C20 | 0.01 | 0.08 | 2.13 | 0.44 | 10.39 | 1.33 |
| C21 | 0.00 | 0.03 | 1.07 | 0.28 | 11.23 | 1.99 |
| C22 | 0.00 | 0.00 | 0.52 | 0.17 | 10.14 | 2.35 |
| C23 | 0.00 | 0.00 | 0.24 | 0.09 | 8.47 | 2.37 |
| C24 | 0.00 | 0.00 | 0.10 | 0.05 | 6.12 | 2.09 |
| C25 | 0.00 | 0.00 | 0.05 | 0.03 | 4.76 | 1.87 |
| C26 | 0.00 | 0.00 | 0.02 | 0.02 | 3.63 | 1.27 |
| C27 | 0.00 | 0.00 | 0.00 | 0.14 | 2.54 | 1.00 |
| C28 | 0.00 | 0.00 | 0.00 | 0.00 | 1.82 | 0.82 |
| C29 | 0.00 | 0.00 | 0.00 | 0.00 | 1.36 | 0.64 |
| C30 | 0.00 | 0.00 | 0.00 | 0.00 | 0.97 | 0.51 |
| C31 | 0.00 | 0.00 | 0.00 | 0.00 | 0.73 | 0.45 |
| C32 | 0.00 | 0.00 | 0.00 | 0.00 | 0.62 | 0.41 |
| C33 | 0.00 | 0.00 | 0.00 | 0.00 | 0.52 | 0.33 |
| C34 | 0.00 | 0.00 | 0.00 | 0.00 | 0.47 | 0.33 |
| C35 | 0.00 | 0.00 | 0.00 | 0.00 | 0.37 | 0.32 |
| C36 | 0.00 | 0.00 | 0.00 | 0.00 | 0.37 | 0.34 |
| C37 | 0.00 | 0.00 | 0.00 | 0.00 | 0.33 | 0.29 |
| C38 | 0.00 | 0.00 | 0.00 | 0.00 | 0.29 | 0.28 |
| C39 | 0.00 | 0.00 | 0.00 | 0.00 | 0.25 | 0.25 |
| C40 | 0.00 | 0.00 | 0.00 | 0.00 | 0.23 | 0.22 |
| C41 | 0.00 | 0.00 | 0.00 | 0.00 | 0.20 | 0.19 |
| C42 | 0.00 | 0.00 | 0.00 | 0.00 | 0.12 | 0.25 |
| C43 | 0.00 | 0.00 | 0.00 | 0.00 | 0.10 | 0.25 |
| C44+ | 0.00 | 0.00 | 0.00 | 0.00 | 0.63 | 1.82 |
| Sum | 87.73 | 12.27 | 90.22 | 9.78 | 76.69 | 23.31 |

The distilled products are rich in normal paraffins, and the overhead fraction has a high cetane index indicating its good potential as a diesel fuel.

The conversion of the waxy feedstock to lighter products was done with a high preservation of the structure of the normal paraffins. The selectivity of the normal paraffin conversion is defined as the ratio of the n-C₂₂ and lighter paraffins produced to the n-C₂₃ and heavier normal paraffins in the feed. n-C₂₂ was chosen as a reference since it corresponds to a boiling point of about 700° F.

$$N \text{-} Paraffin Selectivity = \frac{net n \text{-} C 22 formed in the product \times 100}{net n \text{-} C 23 consumed in the feed} = \frac{( 87.49 \times 0.191 + 89.90 \times 0.706 + 10.3 \times 0.4174 - 23.25 ) \times 100}{66.08 - ( 0.41 \times 0.706 + 34.95 \times 0.103 )} \\$$

For this product the n-Paraffin Selectivity is approximately=98.4% where the yields of the products on the basis of the feed are taken as the yields from the D1160 distillation. The amount of light gases produced is small.

The whole liquid product from hours 666 were solvent dewaxed by the following procedure:

### Laboratory Solvent Dewaxing Procedure

The waxy sample was heated until just above its pour point. 100 grams were poured into a tared 1-liter glass bleaker on a balance and weighed to two decimal places. 200 mL of toluene, and then 200 mol of methyl ethyl ketone were added. The sample was stirred gently until dissolved. Once completely dissolved, the sample in the beaker was covered with a piece of aluminum foil and placed in a freezer which had been preset to the desired temperature (−10° F.) and allowed to sit undisturbed overnight.

The filtration was done in a compartment in the freezer that is equipped with a vacuum line and toggle switch. The filtration assembly consisted of a 186-mm Büchner funnel atop a 2-liter filtering flask. All equipment, including the filtration assembly spatulas, additional ketone etc, was stored in the freezer so that they are at thermal equilibrium.

A No. 4 Whatman filter paper (18.5 cm diameter) was placed in the Buchner funnel. A heavy-duty vacuum hose was attached to the vacuum line in the freezer.

Filtration Procedure:

1. The filter paper was prewetted with 20 ml of cold ketone.
2. The toggle switch to the vacuum was opened.
3. The sample in the beaker was stirred with the spatula and quantitatively transferred to the funnel. The sides of the beaker were scraped to remove additional sample.
4. The sides of the beaker were rinsed with 200 ml of cold ketone.
5. The freezer was closed and the mixture was allowed to filter.
6. Cracks in the filter cake were smoothed over with a spatula to provide a smooth surface.
7. 200 ml of cold ketone were poured over the sample to rinse residual oil.
8. Steps 6 and 7 were repeated.
   Wax Recovery Procedure:

The filter cake was allowed to dry, and the vacuum disconnected. The wax was transferred to a pretared jar. 100 mL of boiling toluene was poured around the rim of the filter to dissolve all remaining wax. The toluene was collected in the jar. To evaporate the solvent, the jar was placed on a hot plate under low heat and in contact with a gentile stream of nitrogen. After the solvent has been removed, the weight of the recovered wax is determined.

Oil Recovery Procedure:

The filtrate was poured into a tared 1-liter round bottom flask and then stripped using a rotary evaporator equipped with a nitrogen stream. The oil-solvent mixture was heated to 120° C. in an oil bath and stripped for a minimum of four hours. The weight of the recovered oil was then determined.

Because of the large amount of light material, recovered wax and dewaxed oil were low. The properties of the dewaxed oil and wax are shown in Table 14.

TABLE 14
Properties of Dewaxed Oil and Residual Wax from Hours 666

| | WLP | DWO | Wax |
|---|---|---|---|
| Wt. % | 100 | 36.5 | 3.2 |
| API Gravity | 54.5 | 50.2 | |
| Vis@40° C., cSt. | | 3.366 | |
| Vis@100° C., cSt. | | 1.296 | |
| VI | | | |
| Pour Point, ° C. | | 9 | |
| Cloud Point, ° C. | | 12 | |
| D2887 Distil., ° F. by | | | |
| wt % | | | |
| 0.5/5 | 95/207 | 419/456 | |
| 10/30 | 257/383 | 485/523 | |
| 50/ | 454/ | 552/ | |
| 70/90 | 522/623 | 580/629 | |
| 95/99 | 670/771 | 670/734 | |

### COMPARATIVE EXAMPLE 20

### Tests on Fischer Tropsch Wax Without Added Hydrogen

Following Example 15, the Pt/B-SSZ-58 catalyst was left in the microunit at 250 psig and temperatures in excess of 725° F. but the hydrogen was replaced with nitrogen gas. The catalyst activity for normal paraffin conversion appeared to halt immediately, and increasing the temperature did not result in any significant conversion.

After 29 hours at 250 psig, temperatures in excess of 725° F. and in the absence of hydrogen, hydrogen was re-introduced and nitrogen gas flow terminated. The catalyst's activity was rapidly restored to its initial value as determined by the appearance of the product GC traces. This demonstrates the remarkable tolerance of the catalyst towards hydrogen interruption. This is an advantage over conventional hydrocracking catalysts.

### EXAMPLE 21

### Tests on a n-Paraffin Containing Petroleum Feedstock

Following Example 20 a waxy hydrocracked Chinese feedstock was processed over the Pt/B-SSZ-58 catalyst. The catalyst was very stable at moderate conversion levels and low pressure (below 300 psig) while giving good yields of distillate fuel. The conversion increased as the pressure decreased. Properties of the feedstock are shown in Table 10. Yields from various operating conditions are shown in Table 15, and analyses of the products from D-1160 distillation of the products from 1362 and 1434 hours are shown in Table 16.

TABLE 15
Hydroconversion of Daqing 650N With Pt/B-SSZ-58

| | Run Hours | | | | | |
|---|---|---|---|---|---|---|
| | 1242 | 1338 | 1362 | 1386 | 1410 | 1434 |
| Temp., ° F. | 755 | 755 | 755 | 745 | 745 | 745 |
| WHSV | 0.99 | 0.96 | 0.97 | 0.96 | 0.97 | 0.94 |
| Tot. P, psig | 1928 | 272 | 271 | 273 | 272 | 272 |
| Gas Rate, SCFB | 2958 | 3055 | 3038 | 3064 | 3040 | 3130 |
| Conv <650° F., Wt % | 13.55 | 31.94 | 32.64 | 29.05 | 29.47 | 30.59 |
| Yields, Wt % | | | | | | |
| Methane | 0.03 | 0.12 | 0.14 | 0.09 | 0.10 | 0.11 |
| Ethane | 0.04 | 0.20 | 0.23 | 0.14 | 0.15 | 0.17 |
| Propane | 0.16 | 0.97 | 1.07 | 0.68 | 0.74 | 0.82 |
| i-Butane | 0.02 | 0.10 | 0.10 | 0.06 | 0.07 | 0.08 |
| n-Butane | 0.27 | 1.55 | 1.54 | 1.04 | 1.08 | 1.22 |
| C₅-180° F. | 0.92 | 4.29 | 5.24 | 3.73 | 3.84 | 4.15 |
| 180-250° F. | 0.49 | 2.32 | 2.31 | 1.78 | 1.77 | 1.85 |
| 250-550° F. | 7.14 | 19.88 | 19.55 | 18.61 | 18.63 | 19.18 |
| 550-700° F. | 7.11 | 4.43 | 4.35 | 4.64 | 4.82 | 4.75 |
| 700° F.+ | 84.91 | 67.78 | 67.15 | 70.72 | 70.37 | 69.28 |
| D2887 Dist, by Wt % | | | | | | |
| St/5% | 99/472 | 32/256 | 32/256 | 82/275 | 152/278 | 94/258 |
| 10/30% | 598/854 | 326/718 | 326/720 | 347/768 | 363/765 | 343/751 |
| 50% | 943/ | 924/ | 924/ | 934/ | 935/ | 933/ |
| 70/90% | 980/1021 | 974/1018 | 974/1018 | 978/1020 | 978/1021 | 979/1021 |
| 95/99% | 1042/1077 | 1039/1074 | 1039/1074 | 1041/1075 | 1043/1079 | 1043/1078 |

TABLE 16
Analysis of the Products from Hydroconversion of Daqing 650N With Pt/B-SSZ-58

| | Run Hours | | | | | | | | | | | |
|---|---|---|---|---|---|---|---|---|---|---|---|---|
| | 1362 | | | | | | 1434 | | | | | |
| | Trap | | Overhead | | Bottoms | | Trap | | Overhead | | Bottom | |
| API Gravity | 61.9 | | 44.1 | | 28.1 | | 62.3 | | 46.2 | | 29.2 | |
| Volume, % | 19.5 | | 10.9 | | 69.6 | | 15.8 | | 12.8 | | 71.4 | |
| Weight, % | 16.8 | | 10.4 | | 72.8 | | 13.6 | | 12.0 | | 74.4 | |
| Cetane | | | 60 | | | | | | 64 | | | |
| Index | | | | | | | | | | | | |
| | | Non | | | | | | | | | | |
| n-Paraffin | | n- | | Non | n- | Non | | Non n- | | Non n- | | |
| Analysis, wt % | n-Par | Par | n-Par | n-Par | Par | n-Par | n-Par | Par | n-Par | Par | n-Par | Non n-Par |
| C4 | 0.2 | 0.00 | 0.00 | 0.00 | 0.00 | 0.00 | 0.17 | 0.00 | 0.00 | 0.00 | 0.00 | 0.00 |
| C5 | 0.97 | 0.11 | 0.00 | 0.00 | 0.00 | 0.00 | 0.92 | 0.09 | 0.00 | 0.00 | 0.00 | 0.00 |
| C6 | 2.88 | 4.60 | 0.00 | 2.36 | 0.00 | 0.00 | 2.80 | 3.89 | 0.00 | 0.00 | 0.00 | 0.00 |
| C7 | 6.60 | 1.24 | 0.00 | 0.02 | 0.00 | 0.00 | 6.50 | 1.14 | 0.01 | 0.00 | 0.00 | 0.00 |
| C8 | 11.45 | 2.87 | 0.01 | 0.02 | 0.00 | 0.00 | 11.39 | 2.78 | 0.02 | 0.01 | 0.00 | 0.00 |
| C9 | 13.52 | 4.15 | 0.04 | 0.04 | 0.00 | 0.00 | 13.68 | 3.64 | 0.05 | 0.07 | 0.00 | 0.00 |
| C10 | 13.36 | 5.08 | 0.44 | 0.11 | 0.00 | 0.00 | 14.09 | 4.49 | 0.46 | 0.11 | 0.00 | 0.00 |
| C11 | 10.76 | 5.71 | 2.84 | 0.59 | 0.00 | 0.00 | 12.40 | 5.08 | 2.84 | 0.51 | 0.00 | 0.00 |
| C12 | 4.97 | 5.20 | 8.48 | 2.47 | 0.00 | 0.00 | 6.49 | 4.28 | 8.39 | 1.93 | 0.00 | 0.00 |
| C13 | 1.28 | 2.96 | 10.56 | 5.80 | 0.00 | 0.00 | 1.87 | 2.22 | 11.26 | 4.36 | 0.00 | 0.00 |
| C14 | 0.27 | 0.97 | 8.48 | 7.42 | 0.00 | 0.01 | 0.45 | 0.75 | 10.30 | 5.88 | 0.00 | 0.04 |
| C15 | 0.07 | 0.26 | 5.58 | 7.87 | 0.01 | 0.00 | 0.12 | 0.28 | 8.12 | 6.26 | 0.00 | 0.00 |
| C16 | 0.02 | 0.13 | 2.92 | 7.67 | 0.02 | 0.07 | 0.04 | 0.10 | 5.63 | 6.31 | 0.01 | 0.04 |
| C17 | 0.01 | 0.05 | 1.31 | 6.66 | 0.08 | 0.08 | 0.01 | 0.08 | 3.31 | 6.00 | 0.00 | 0.15 |
| C18 | 0.00 | 0.05 | 0.56 | 5.72 | 0.15 | 0.22 | 0.01 | 0.03 | 1.57 | 5.06 | 0.05 | 0.16 |
| C19 | 0.00 | 0.04 | 0.32 | 4.06 | 0.14 | 0.33 | 0.00 | 0.02 | 0.03 | 4.75 | 0.06 | 0.24 |
| C20 | 0.00 | 0.00 | 0.15 | 2.64 | 0.11 | 0.32 | 0.00 | 0.19 | 0.24 | 2.20 | 0.05 | 0.22 |
| C21 | 0.00 | 0.00 | 0.04 | 1.65 | 0.04 | 0.34 | 0.00 | 0.00 | 0.06 | 1.58 | 0.02 | 0.24 |
| C22 | 0.00 | 0.00 | 0.04 | 1.19 | 0.04 | 0.39 | 0.00 | 0.00 | 0.03 | 1.05 | 0.03 | 0.27 |
| C23 | 0.00 | 0.00 | 0.02 | 0.79 | 0.04 | 0.37 | 0.00 | 0.00 | 0.02 | 0.67 | 0.03 | 0.24 |
| C24 | 0.00 | 0.00 | 0.01 | 0.51 | 0.03 | 0.32 | 0.00 | 0.00 | 0.01 | 0.42 | 0.03 | 0.21 |
| C25 | 0.00 | 0.00 | 0.00 | 0.30 | 0.08 | 0.41 | 0.00 | 0.00 | 0.02 | 0.27 | 0.01 | 0.25 |
| C26 | 0.00 | 0.00 | 0.01 | 0.19 | 0.05 | 0.48 | 0.00 | 0.00 | 0.00 | 0.12 | 0.03 | 0.46 |
| C27 | 0.00 | 0.00 | 0.00 | 0.12 | 0.07 | 0.22 | 0.00 | 0.00 | 0.00 | 0.09 | 0.10 | 0.16 |
| C28 | 0.00 | 0.00 | 0.00 | 0.00 | 0.08 | 0.40 | 0.00 | 0.00 | 0.00 | 0.00 | 0.06 | 0.26 |
| C29 | 0.00 | 0.00 | 0.00 | 0.00 | 0.04 | 0.49 | 0.00 | 0.00 | 0.00 | 0.00 | 0.03 | 0.38 |
| C30 | 0.00 | 0.00 | 0.00 | 0.00 | 0.01 | 0.57 | 0.00 | 0.00 | 0.00 | 0.00 | 0.01 | 0.45 |
| C31 | 0.00 | 0.00 | 0.00 | 0.00 | 0.02 | 0.32 | 0.00 | 0.00 | 0.00 | 0.00 | 0.03 | 0.26 |
| C32 | 0.00 | 0.00 | 0.00 | 0.00 | 0.06 | 0.27 | 0.00 | 0.00 | 0.00 | 0.00 | 0.04 | 0.22 |
| C33 | 0.00 | 0.00 | 0.00 | 0.00 | 0.05 | 0.27 | 0.00 | 0.00 | 0.00 | 0.00 | 0.04 | 0.18 |
| C34 | 0.00 | 0.00 | 0.00 | 0.00 | 0.12 | 0.51 | 0.00 | 0.00 | 0.00 | 0.00 | 0.08 | 0.66 |
| C35 | 0.00 | 0.00 | 0.00 | 0.00 | 0.11 | 1.36 | 0.00 | 0.00 | 0.00 | 0.00 | 0.08 | 1.68 |
| C36 | 0.00 | 0.00 | 0.00 | 0.00 | 0.15 | 3.13 | 0.00 | 0.00 | 0.00 | 0.00 | 0.12 | 3.19 |
| C37 | 0.00 | 0.00 | 0.00 | 0.00 | 0.12 | 5.24 | 0.00 | 0.00 | 0.00 | 0.00 | 0.11 | 5.17 |
| C38 | 0.00 | 0.00 | 0.00 | 0.00 | 0.20 | 10.02 | 0.00 | 0.00 | 0.00 | 0.00 | 0.17 | 8.79 |
| C39 | 0.00 | 0.00 | 0.00 | 0.00 | 0.03 | 12.84 | 0.00 | 0.00 | 0.00 | 0.00 | 0.03 | 11.09 |
| C40 | 0.00 | 0.00 | 0.00 | 0.00 | 0.20 | 13.16 | 0.00 | 0.00 | 0.00 | 0.00 | 0.17 | 11.63 |
| C41 | 0.00 | 0.00 | 0.00 | 0.00 | 0.08 | 12.63 | 0.00 | 0.00 | 0.00 | 0.00 | 0.07 | 11.52 |
| C42 | 0.00 | 0.00 | 0.00 | 0.00 | 0.13 | 10.74 | 0.00 | 0.00 | 0.00 | 0.00 | 0.08 | 10.48 |
| C43 | 0.00 | 0.00 | 0.00 | 0.00 | 0.14 | 8.95 | 0.00 | 0.00 | 0.00 | 0.00 | 0.06 | 8.55 |
| C44+ | 0.00 | 0.00 | 0.00 | 0.00 | 0.32 | 12.80 | 0.00 | 0.00 | 0.00 | 0.00 | 0.28 | 20.93 |
| Sum | 66.39 | 33.6 | 41.80 | 58.2 | 2.75 | 97.25 | 70.94 | 29.06 | 52.36 | 47.64 | 1.89 | 98.11 |

These results show that a high cetane index product is generated, along a bottoms product that is depleted in normal paraffins. The bottoms product was solvent dewaxed to give a dewaxed oil with the properties shown in Table 17. The n-paraffins selectivity for this feed is about 61%. Apparently, in feedstocks containing modest levels of n-paraffins, some of the n-paraffins hydroconvert to form other products—possibly isoparaffins, cycloparaffins, or alkyl groups. Thus, when n-paraffin products are desired, the feedstock must contain >5 wt % n-paraffins, preferably >50 wt % n-paraffins, and more preferably >80 wt % n-paraffins.

TABLE 17
Dewaxing Results and Lubricant Properties of the 650° F.+ Products from
Hydroconversion of Daqing 650N With Pt/B-SSZ-58

| | Run Hours | | | | | |
|---|---|---|---|---|---|---|
| | 1338-1362 | | | 1386-1434 | | |
| | WLP | DWO | Wax | WLP | DWO | Wax |
| Wt. % | 100 | 70.4 | 10.5 | 100 | 73.8 | 11.2 |
| API | 36.2 | 30.3 | | 35.9 | 31.2 | |
| Gravity | | | | | | |
| Vis @ | | 46.26 | | | 41.72 | |
| 40 C., | | | | | | |
| cSt. | | | | | | |
| Vis @ | | 7.312 | | | 6.90 | |
| 100 C., | | | | | | |
| cSt. | | | | | | |
| VI | | 120 | | | 123 | |
| Pour | | −5 | | | −3 | |
| Point, C. | | | | | | |
| Cloud | | −3 | | | −2 | |
| Point, C. | | | | | | |
| D2887 | | | | | | |
| Distil., | | | | | | |
| ° F. by | | | | | | |
| wt % | | | | | | |
| 0.5/5 | 129/256 | 406/488 | | 152/278 | 397/485 | |
| 10/30 | 326/718 | 584/853 | | 363/765 | 547/852 | |
| 50/ | 924/ | 935/ | | 935/ | 942/ | |
| 70/90 | 974/1018 | 976/010 | | 978/1021 | 976/1010 | |
| 95/99 | 1039/1074 | 1025/1044 | | 1043/1079 | 1025/1043 | |

These results show that a high viscosity index product can be made by a combination of a n-paraffin selective hydroconversion process and a solvent dewaxing process.

### EXAMPLE 22

### Preparation of Large Sample of Pt/B-SSZ-33

Borosilicate SSZ-33 was synthesized as follows following the procedure in Example 14.

Pt/B-SSZ-33 was made according to the following procedure. The B-SSZ-33 sample was calcined to remove the template. Calcination was done as follows. A thin bed of material is heated in a muffle furnace from room temperature to 120° C. at a rate of 1° C. per minute and held at 120° C. for 1 hour. The temperature is then ramped up to 540° C. at the same rate and held at this temperature for 5 hours, after which it is increased to 595° C. and held there for another 5 hours. The atmosphere for calcination is nitrogen at a rate of 20 standard cubic feet per minute with a small amount of air being bled into the flow. The pore volume of the calcined B-SSZ-33 was 0.21 cm3/g. The Si/B molar ratio of the product was 18.

The calcined sample was impregnated by adding an aqueous ammonium nitrate solution (0.1506 gm Pt(NH₃)₄(NO₃)₂ in 35.3 gm deionized water) to 15.15 gm zeolite at the dry weight at 350° C. After 48 hours at room temperature, the mixture was dried in a vacuum oven at 110° C. for 3 hours. Then the sample was calcined in air as follows: heat from room temperature to 120° C. in 1 hour, keep at 120° C. for 1 hour, heat from 120° C. to 300° C. in 3 hours, keep at 300° C. for 5 hours, then cooled down to room temperature, resulting in a calcined Pt/B-SSZ-33 catalyst containing 0.5 wt. % Pt on the dry zeolite. The Pt/B-SSZ-33 was then pelletized to 24-42 mesh for use of catalytic testing.

### EXAMPLE 23

### Catalytic Testing of Pt/B-SSZ-33

The catalyst from Example 22 was tested in a microunit using the Daqing feedstock described in Example 19. Results are shown in Table 18.

TABLE 18
HCR of Daqing 650N With Pt/B-SSZ-33

| | | | | | | |
|---|---|---|---|---|---|---|
| Hours | 402 | 426 | 450 | 570 | 594 | 618 |
| Temp., ° F. | 770 | 770 | 770 | 790 | 790 | 790 |
| WHSV | 1.12 | 1.12 | 1.10 | 1.11 | 1.11 | 1.11 |
| Tot. P, psig | 394 | 396 | 396 | 390 | 392 | 393 |
| Gas Rate, SCFB | 3066 | 3064 | 3106 | 3096 | 3091 | 3091 |
| Conv <650 F, Wt % | 33.15 | 32.51 | 32.44 | 53.72 | 50.97 | 51.90 |
| HCR k, hr−1 | 0.45 | 0.44 | 0.43 | 0.86 | 0.79 | 0.81 |
| No Loss Yields, Wt % | | | | | | |
| Methane | 0.16 | 0.15 | 0.16 | 0.30 | 0.28 | 0.27 |
| Ethane | 0.19 | 0.17 | 0.17 | 0.37 | 0.37 | 0.34 |
| Propane | 0.69 | 0.59 | 0.60 | 1.21 | 1.29 | 1.13 |
| i-Butane | 0.25 | 0.22 | 0.22 | 0.47 | 0.54 | 0.46 |
| n-Butane | 0.72 | 0.66 | 0.66 | 1.42 | 1.66 | 1.37 |
| C₅- | 2.80 | 3.13 | 2.72 | 5.17 | 4.76 | 4.55 |
| 180° F. | | | | | | |
| 180-250° F. | 1.50 | 1.76 | 1.74 | 2.90 | 2.76 | 3.33 |
| 250-550° F. | 17.34 | 16.33 | 16.55 | 29.76 | 28.41 | 29.33 |
| 550-700° F. | 14.37 | 13.72 | 14.37 | 16.48 | 14.56 | 15.38 |
| 700° F.+ | 62.41 | 63.71 | 63.25 | 42.48 | 45.94 | 44.39 |
| D2887 Distil., ° F. by wt % | | | | | | |
| St/5% | 567/623 | 602/671 | 608/669 | 615/666 | 612/671 | 607/664 |
| 10/30% | 661/815 | 709/852 | 704/848 | 691/790 | 701/808 | 691/796 |
| 50% | 920/ | 929/ | 929/ | 879/ | 894/ | 886/ |
| 70/90% | 962/1007 | 967/1009 | 968/1011 | 947/999 | 954/1003 | 951/1001 |
| 95/99% | 1029/1058 | 1032/1060 | 1036/1069 | 1018/1055 | 1022/1059 | 1021/1057 |

The catalyst proved to be remarkably stable when operated at high conversions and low pressures. Yields of C₄ gases were low as were the yields of naphtha products. The catalyst was most selective for distillate products.

The overhead product collected from the on-line distillation from 570-618 hours was distilled by D-1160 into rough cuts to simulate naphtha, jet and diesel. The products are shown in Table 19. The jet cut has an excellent smoke point.

TABLE 19
Properties of Cuts from D1160 Distillation of the Blended Stripper Overhead Products from 570-618
Hours

| | Feed | Light Ends | Overhead | Bottoms |
|---|---|---|---|---|
| API Gravity | | 62.7 | 48.8 | 44.4 |
| Volume, % | 100 | 13.17 | 66.51 | 20.32 |
| Weight, % | 100 | 12.29 | 66.90 | 20.82 |
| Smoke Point, mm | | | 40 | |
| Freeze Point, C. | | | −21 | |
| Cloud Point, C. | | | | 2 |
| SFC Aromatics | | | | |
| Mono-aromatics, wt % | | | 1.8 | |
| PNA's, wt % | | | 0.0 | |
| Total Aromatics, wt % | | | 1.8 | |

| Paraffin Analysis, wt % | n-P | Non-n-P | n-P | Non-n-P | n-P | Non-n-P | n-P | Non-n-P |
|---|---|---|---|---|---|---|---|---|
| C4 | | 0.22 | | 0.13 | | | | |
| C5 | 0.58 | 0.02 | 1.43 | 0.23 | 0.01 | | | |
| C6 | 1.28 | 0.75 | 5.09 | 2.31 | 0.00 | | | |
| C7 | 2.39 | 1.87 | 10.21 | 6.92 | 0.00 | | | |
| C8 | 2.94 | 3.48 | 12.15 | 13.01 | 0.02 | 0.67 | | |
| C9 | 2.89 | 4.28 | 9.24 | 13.55 | 1.08 | 4.65 | | |
| C10 | 3.05 | 4.90 | 4.53 | 8.95 | 4.03 | 8.20 | | |
| C11 | 3.19 | 5.34 | 1.97 | 4.11 | 5.75 | 9.23 | | |
| C12 | 2.85 | 5.59 | 0.88 | 2.00 | 5.55 | 9.76 | 0.01 | |
| C13 | 2.53 | 5.87 | 0.45 | 1.09 | 5.60 | 9.52 | 0.03 | 0.01 |
| C14 | 2.31 | 6.03 | 0.23 | 0.61 | 5.02 | 9.02 | 0.66 | 0.42 |
| C15 | 2.05 | 6.04 | 0.13 | 0.36 | 4.08 | 6.33 | 3.19 | 3.51 |
| C16 | 1.72 | 5.62 | 0.08 | 0.14 | 2.74 | 4.04 | 6.45 | 10.98 |
| C17 | 1.46 | 5.36 | 0.04 | 0.09 | 1.67 | 1.57 | 9.46 | 18.10 |
| C18 | 1.13 | 5.13 | 0.01 | 0.03 | 0.55 | 0.40 | 5.88 | 20.66 |
| C19 | 0.78 | 4.62 | 0.00 | 0.01 | 0.14 | 0.07 | 3.09 | 10.40 |
| C20 | 0.28 | 2.80 | 0.00 | 0.02 | 0.02 | 0.26 | 0.80 | 5.16 |
| C21 | 0.03 | 0.62 | 0.00 | 0.00 | 0.00 | 0.00 | 0.08 | 0.92 |
| C22 | 0.00 | 0.02 | 0.00 | 0.00 | 0.00 | 0.00 | 0.01 | 0.12 |
| C23 | 0.00 | 0.00 | 0.00 | 0.00 | 0.00 | 0.00 | 0.00 | 0.02 |
| C24 | 0.00 | 0.00 | 0.00 | 0.00 | 0.00 | 0.00 | 0.00 | 0.04 |
| Sum | 31.45 | 68.54 | 46.45 | 53.55 | 36.28 | 63.73 | 29.66 | 70.34 |

The products contained significant quantities of n-paraffins, but since the SSZ-33 catalyst contains 12-ring structures, it converted not only n-paraffins in the feed but non-n-paraffins (isoparaffins and cyclic compounds).

The bottoms product from the on-line distillation was solvent dewaxed and the products analyzed as shown in Table 20.

TABLE 20
Analysis of Stripper Bottoms Product Blend from 570-618 hours

| | SLP | DWO | Wax |
|---|---|---|---|
| Wt. % | 100 | 86.1 | 13.9 |
| API Gravity | 30.6 | 30.4 | |
| N, ppm | 0.4 | | |
| S, ppm | <2.0 | | |
| Vis@40 C, cSt. | 48.27 | 51.12 | |
| Vis@100 C, cSt. | 7.456 | 7.48 | |
| VI | 117 | 108 | |
| Pour Point, C | +20 | −14 | |
| Cloud Point, C | +24 | −13 | |
| D2887 Distil., ° F. by wt % | | | |
| 0.5/5 | 623/665 | 637/683 | |
| 10/30 | 691/798 | 715/815 | |
| 50/ | 893/ | 904/ | |
| 70/90 | 953/1012 | 967/1020 | |
| 95/99 | 1046/1187 | 1046/1145 | |

The dewaxed oil gave a high VI lubricant. The VI of the product from 450 hours of operation was 114.

The original stripped liquid product (SLP), the dewaxed oil (DWO) and the wax were further analyzed for normal paraffin content as shown in Table 21.

TABLE 21
Paraffin Analysis of Stripper Bottoms Product Blend from 570-618 hours

| Weight, % | | | | | | |
|---|---|---|---|---|---|---|
| n-Paraffin | SLP (Blend 2) | | DWO | | Wax | |
| Analysis, | 100 | | 86.1 | | 13.9 | |
| wt % | n-P | Non-n-P | n-P | Non-n-P | n-P | Non-n-P |
| C17− | 0.00 | 0.00 | 0.00 | 0.00 | 0.07 | 0.00 |
| C17 | 0.00 | 0.00 | 0.00 | 0.01 | 0.02 | 0.00 |
| C18 | 0.00 | 0.00 | 0.00 | 0.01 | 0.02 | 0.01 |
| C19 | 0.05 | 0.10 | 0.03 | 0.19 | 0.14 | 0.01 |
| C20 | 0.28 | 1.50 | 0.08 | 1.84 | 1.53 | 0.03 |
| C21 | 0.41 | 3.38 | 0.05 | 4.28 | 2.82 | 0.17 |
| C22 | 0.32 | 3.61 | 0.02 | 4.78 | 2.53 | 0.34 |
| C23 | 0.23 | 3.60 | C23+ | 88.71 | 2.01 | 0.47 |
| C24 | 0.18 | 3.43 | | | 1.50 | 0.51 |
| C25 | 0.14 | 3.38 | | | 1.20 | 0.54 |
| C26 | 0.12 | 3.49 | | | 0.95 | 0.59 |
| C27 | 0.09 | 3.55 | | | 0.80 | 0.69 |
| C28 | 0.12 | 3.97 | | | 0.66 | 0.81 |
| C29 | 0.14 | 3.56 | | | 0.53 | 1.04 |
| C30 | 0.14 | 2.73 | | | 0.44 | 1.13 |
| C31 | 0.10 | 3.34 | | | 0.34 | 1.46 |
| C32 | 0.07 | 3.02 | | | 0.30 | 1.66 |
| C33 | 0.05 | 3.34 | | | 0.27 | 1.99 |
| C34 | 0.04 | 3.37 | | | 0.20 | 2.42 |
| C35 | C35+ | 48.16 | | | 0.20 | 3.05 |
| C36 | | | | | 0.24 | 3.28 |
| C37 | | | | | 0.25 | 4.74 |
| C38 | | | | | 0.17 | 6.43 |
| C38+ | | | | | | 51.44 (mostly |
| | | | | | | non-n P) |
| Sum | 2.48 | 97.53 | 0.18 | 99.82 | 17.19 | 82.81 |

### EXAMPLE 24

### Preparation and Testing of Pt/B-SSZ-60

SSZ-60 is a one dimensional 10-ring zeolite. The preparation of B-SSZ-60 is described in U.S. Pat. No. 6,620,401, issued Sep. 16, 2003 to Elomari, (Examples 3 and 4) and U.S. Pat. No. 6,540,906, issued Apr. 1, 2003 to Elomari. Example 8 in U.S. Pat. No. 6,620,401 describes the testing of a Pt/Al-SSZ-60 that was prepared by conversion of the B-SSZ-60 into an aluminosilicate zeolite. The results show that aluminum incorporation generates unwanted acidity as shown by high product i/n ratios.

A Pt/B-SSZ-60 was prepared by the same method used in Example 2. The catalyst was tested using n-C₁₆ as described in Example 1. Results were obtained at conversions near 80% and the linearly interpolated value at 80% conversion was derived. Results are shown in Table 22.

### EXAMPLE 25

### Preparation and Testing of Pt/B-SSZ-70

SSZ-70 is believed to be a four dimensional 10-ring zeolite—it has two sets of 2-dimensional 10 ring pores. B-SSZ-70 was made by mixing tetraethylorthosilicate and triethylborate at a Si/B molar ratio of 18:1 with a 1 molar solution of diisopropylimidazolium to give a Si/template molar ratio of 2 and then allowed to evaporate in a hood for several days to remove ethanol. The contents of the tared reactor (Teflon cup for a Parr 4745 Stainless steel reactor) were then adjusted with water to achieve the H₂O/Si molar ratio of 18. Finally 50% HF was added dropwise with a plastic spatula being used to stir the contents as it gels to give a Si/F molar ratio of 2. The reaction is then heated to 150° C. for 80 days.

A Pt/B-SSZ-70 was prepared by the same method used in Example 2. The catalyst was tested using n-C₁₆ as described in Example 1. Results were obtained at conversions near 80% and the linearly interpolated value at 80% conversion was derived. Results are shown in Table 22.

TABLE 22
Comparison of Catalysts at 80% Hydroconversion of n-C₁₆ to Products
Less than C₁₆

| | Example | | |
|---|---|---|---|
| | 2 | 24 | 25 |
| Catalyst | PtBZSM5 | PtBSSZ60 | PtBSSZ70 |
| Sieve Structure | 3D 10R | 2D 10R | 4D 10R |
| Micropore vol. cm³/g | | | |
| SEM Size, μ | | | |
| Temp., ° F. | 577 | 653 | 682 |
| nC₁₆ Conv | 80.00 | 80.00 | 80.00 |
| i/n Ratios | | | |
| C₄ i/n | 0.00 | 0.01 | 0.01 |
| C₅ i/n | 0.00 | 0.02 | 0.01 |
| C₆ i/n | 0.00 | 0.04 | 0.02 |
| C₇ i/n | 0.00 | 0.02 | 0.02 |
| C₈ i/n | 0.00 | 0.02 | 0.02 |
| C₉ i/n | 0.00 | 0.01 | 0.02 |
| C₁₀ i/n | 0.00 | 0.02 | 0.03 |
| C₁₁ i/n | 0.00 | 0.01 | 0.04 |
| C₁₂ i/n | 0.00 | 0.01 | 0.02 |
| C₁₃ i/n | 0.00 | 0.02 | 0.01 |
| C₄-C₁₃ i/n | 0.00 | 0.02 | 0.02 |
| Yields, wt % | | | |
| C₁ | 0.06 | 0.33 | 0.00 |
| C₂ | 0.26 | 0.71 | 1.05 |
| C₃ | 3.83 | 5.77 | 3.78 |
| C₄s | 6.84 | 8.92 | 6.35 |
| C₅s | 11.69 | 8.82 | 8.69 |
| C₆s | 15.06 | 9.15 | 12.69 |
| C₇-C₁₃ | 61.21 | 63.92 | 64.63 |
| iC16 | 1.06 | 2.38 | 2.83 |
| Unidentified | 0.00 | 0.00 | 0.00 |
| Ratio C₃ + ₄/C₇-₁₃ | 0.18 | 0.23 | 0.16 |
| C₆ i/n low-high | | | |
| Delta | 0.00 | 0.00 | 0.00 |
| Percent change | 0 | 0 | 0 |

These results show that other 10-Ring zeolites can give products with very low i/n ratios, but the B-ZSM-5 material is preferred because of its higher activity.

### EXAMPLE 26

### Preparation and Testing of Pt/B-SSZ-13

Two samples of boron-SSZ-13 were prepared and converted into Pt catalysts. SSZ-13 is an 8-ring zeolite with the Chabazite structure. The aluminum content of the boric acid used in both preparations was measured as <5 ppm. The composition of the Cab-O-Sil M-5 was 3 ppm aluminum and 7 ppm sodium.

### EXAMPLE 26A

This sample of boron-SSZ-13 was made hydrothermally according to the following procedure. In a 600 cc Teflon liner, dissolve 2.15 gm of boric acid in a mixture composed of 134.61 gm of 0.72 molar N,N,N-trimethyl-1-adamantylammonium hydroxide and 41.15 gm of 0.28 molar N,N,N-trimethyl-1-adamantylammonium hydroxide. Add 0.72 gm boron SSZ-13 seeds and 26.13 gm of Cab-O-Sil M-5 (fumed silica—98% SiO₂) with stirring. After thorough mixing, the resulting gel was placed in a 600 cc stirred autoclave and heated at 160° C., 75 rpm for 3 days. The resulting mixture was filtered, washed with deionized H₂O and dried at 95° C. to yield 31.53 gm boron SSZ-13 (X-ray analysis).

The measured boron and aluminum contents are 0.64 wt %. boron and 15 ppm aluminum. The measured sodium content was 187 ppm.

### EXAMPLE 26B

This sample of boron-SSZ-13 was made hydrothermally according to the following procedure. In a 23 cc Teflon liner, add 9.70 gm 0.62 molar N,N,N-trimethyl-1-adamantylammonium hydroxide, 0.12 gm sodium chloride, 0.12 gm boric acid, 1.45 gm Cab-O-Sil M-5 (fumed silica—98% SiO₂) and 0.04 gm boron-SSZ-13 seeds then the mixture was thoroughly mixed. The resulting gel was capped off and placed in a Parr autoclave and heated at 160° C. while rotating at about 43 rpm for 5 days. The resulting mixture was filtered and the obtained solid was thoroughly rinsed with water and air-dried to yield 1.64 gm of boron-SSZ-13 (X-ray analysis).

The measured boron content was 0.63 wt % boron. Based on the aluminum contents of the silicon and boron reagents, the calculated aluminum content should be below 200 ppm, probably below 10 ppm. The measured sodium content was 3569 ppm.

Preparation of the Pt Catalysts.

Platinum impregnation by incipient wetness was carried out by first determining the volume of water necessary to achieve incipient wetness in a known amount of zeolite. This was done by drying a gram of zeolite in a tared 60 cc Pierce bottle at 300° C. for 3 hours then capping it while hot. Upon cooling the dried weigh of the zeolite was determined by weight difference. Then the zeolite-containing Pierce bottle was tared again, and water was introduced by syringe until incipient wetness was achieved. The incipient wetness volume required per gram of dry zeolite was calculated from the weight difference.

For the real platinum impregnation, another fraction of zeolite was dried in a Pierce bottle as before and the volume of water necessary to cause incipient wetness calculated. Next platinum tetraaminedinitrate for 5% Pt loading was dissolved in that volume of water, and the solution was injected into the Pierce bottle by syringe. The catalysts were let to stand overnight then the Pierce bottle was uncapped. The catalysts were dried, then calcined in an oven with flowing air by ramping the temperature at 1° C./hr until 288° C. and then held at this temperature for 3 hours.

The Pt catalysts were tested using n-C₁₆ as described in Example 1. Results were obtained at conversions near 80% and the linearly interpolated value at 80% conversion was derived. Results are shown in Table 23. These demonstrate that 8-ring zeolites can also be used as catalysts for this process. Sample 26B shows lower activity and high product i/n ratios. This is presumably due to the higher sodium content in this sample which reduces activity. Thus, if sodium is added in an attempt to reduce the activity of strongly acidic sites, an excess should be avoided as this will cause a loss in activity. The higher temperatures needed to work with the less active catalyst will result in higher product i/n ratios.

TABLE 23
Test Results for Pt/B-SSZ-13 catalysts

Example

| | 26A | 26B |
|---|---|---|
| Catalyst | PtBSSZ13 | PtBSSZ13 |
| Sieve Structure | 3D 8R | 3D 8R |
| Temp., ° F. | 695 | 736 |
| nC₁₆ Conv | 80.00 | 80.00 |
| i/n Ratios | | |
| C₄ i/n | 0.03 | 0.14 |
| C₅ i/n | 0.03 | 0.11 |
| C₆ i/n | 0.05 | 0.14 |
| C₇ i/n | 0.05 | 0.19 |
| C₈ i/n | 0.07 | 0.26 |
| C₉ i/n | 0.09 | 0.34 |
| C₁₀ i/n | 0.11 | 0.46 |
| C₁₁ i/n | 0.09 | 0.48 |
| C₁₂ i/n | 0.11 | 0.57 |
| C₁₃ i/n | 0.11 | 0.70 |
| C₄-C₁₃ i/n | 0.06 | 0.25 |
| Yields, wt % | | |
| C₁ | 0.93 | 0.98 |
| C₂ | 1.07 | 1.25 |
| C₃ | 5.35 | 5.90 |
| C₄s | 8.25 | 10.01 |
| C₅s | 10.15 | 11.69 |
| C₆s | 11.23 | 11.57 |
| C₇-C₁₃ | 58.47 | 48.28 |
| iC16 | 4.54 | 10.32 |
| Unidentified | 0.00 | 0.00 |
| Ratio C₃ + ₄/C₇-₁₃ | 0.23 | 0.33 |
| C₆ i/n low-high | | |
| Delta | 0.00 | 0.01 |
| Percent change | 0 | 7.69 |

### EXAMPLE 27

### Preparation and Testing of Pt/B-MTT

A zeolite of the MTT structure having 1-dimensional 10-ring pores was prepared. The preparation consisted of two parts: preparation of the seeds, and then preparation of the catalyst.

Preparation of Seeds

In a 23 cc Teflon liner, 0.90 g 1N KOH and 0.70 g N-isopropyl-1,3-propanediamine were dissolved in 10.6 g deionized water. 0.07 g potassium borate tetrahydrate was then dissolved in the mixture, and then 0.90 g Cabosil M-5 was added and mixed to create a uniform suspension. 0.03 g B-MTT seeds from a previous synthesis (Si/B=49) were then added to the mixture. The liner was then capped and sealed in a 23-ml steel Parr autoclave and subsequently placed in an oven with a rotating spit (43 rpm) at 160° C. After four days, the resulting solids were filtered, washed with about 500 mL deionized water, and dried in an oven at 90° C. Powder X-ray diffraction indicated the sample was pure B-MTT. ICP indicated a Si/B molar ratio of 34.

Preparation of B-MTT

In a 23 cc Teflon liner, 0.90 g 0.1N KOH, 10.6 g deionized water, and 0.70 g N-isopropyl-1,3-propanediamine were mixed together. Next 0.11 g potassium borate tetrahydrate was dissolved in the solution. 0.90 g Cabosil M-5 was then added and mixed to create a uniform suspension. 0.03 g of B-MTT seeds were added. The liner was then capped and then sealed in a 23-ml steel Parr autoclave and subsequently placed in an oven with a rotating spit (43 rpm) at 160° C. After seven days, the autoclave was removed and cooled to room temperature. 1.0 g additional KOH was then added to the mixture, which was then resealed and heated for an additional four days. At that point, the resulting solids were filtered, washed with about 500 mL deionized water, and dried in an oven at 90° C. Powder X-ray diffraction indicated the sample was pure B-MTT.

Preparation of the Catalyst

A Pt impregnated catalyst was calcined and prepared following the procedure for Pt-B-SSZ-13 described in Example 26 with the exception that the water pore volume was estimated from results obtained on other samples rather than measured.

Catalyst Testing

The Pt catalysts were tested using n-C₁₆ as described in Example 1. Results were obtained at conversions near 80% and the linearly interpolated value at 80% conversion was derived. Results are shown in Table 24. Pt-B-MTT shows excellent selectivity to heavy products, low i/n ratios, and good activity. This makes it one of the preferred choices.

TABLE 24
Test Results for Pt/B-MTT Catalyst

| Catalyst | Pt/B-MTT |
|---|---|
| Sieve Structure | 1D 10R |
| Temp., ° F. | 667 |
| nC₁₆ Conv | 80.00 |
| i/n Ratios | |
| C₄ i/n | 0.02 |
| C₅ i/n | 0.02 |
| C₆ i/n | 0.02 |
| C₇ i/n | 0.02 |
| C₈ i/n | 0.02 |
| C₉ i/n | 0.02 |
| C₁₀ i/n | 0.02 |
| C₁₁ i/n | 0.03 |
| C₁₂ i/n | 0.04 |
| C₁₃ i/n | 0.06 |
| C₄-C₁₃ i/n | 0.03 |
| Yields, wt % | |
| C₁ | 0.18 |
| C₂ | 0.36 |
| C₃ | 2.68 |
| C₄s | 5.15 |
| C₅s | 6.83 |
| C₆s | 8.52 |
| C₇-C₁₃ | 72.05 |
| iC16 | 4.25 |
| Unidentified | 0.00 |
| Ratio C₃ + ₄/C₇-₁₃ | 0.11 |
| C₆ i/n low-high | |
| Delta | 0.00 |
| Percent change | 0 |

Upgrading of Fischer Tropsch Products

This embodiment describes a preferred method to convert Fischer Tropsch products into a diesel fuel that has a combination of superior yields and properties in comparison to conventional Fischer Tropsch diesel fuels.

Products from the Fischer Tropsch process consist partially of materials already in the diesel boiling range, and partially of heavier materials. In conventional processing, the diesel boiling range material is either hydrotreated and then blended into the diesel pool, or used directly as a pool blend component. Heavy materials are hydrocracked over acidic catalysts to form an isoparaffinic blend component which is blended with the first diesel material. Conventional hydrocracking produces isoparaffins throughout the boiling range of the diesel.

In this preferred embodiment, the heavy material is converted to diesel range materials by n-paraffin selective hydroconversion. The heaviest portions of the diesel fuel which otherwise might have unacceptable cloud points are isomerized. This gives a product with a maximum amount of normal paraffins. Only the heaviest materials are hydroisomerized thus giving an increased amount of normal paraffins and a higher cetane number.

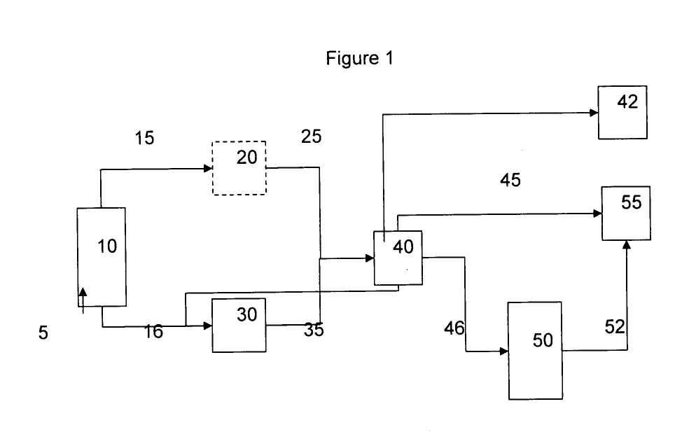

In FIG. 1, a synthesis gas (5) is fed to a slurry bed Fischer Tropsch unit using a supported cobalt catalyst. A vapor phase product is removed from the reactor, cooled and a liquid condensate (15) is recovered. The condensate can be treated in an optional treater (20) to remove oxygenates and saturate olefins. The optional treater can be a conventional hydrotreater using a non-acidic support, or it can be an alumina treater. The latter converts the oxygenates into olefins by dehydration. The treated condensate (25) is sent to a distillation facility (40). The Fischer Tropsch process also produces a waxy product (16) which is fed to a n-paraffin selective hydroconversion reactor (30) to produce an effluent (35). Optionally the waxy product (16) is first hydrotreated to remove impurities such as nitrogen, oxygen, olefins, solids, etc., in a reactor not shown. The hydroconversion reactor uses a 10-ring borosilicate, preferably silica-bound Pt/B-ZSM-5 containing less than 20 weight ppm aluminum, and operates at 250 psig, 1 LHSV, and at a temperature to give 70 percent per pass conversion. The effluent from the hydrocracker is also sent to the distillation facility. A light product, or series of light products, (42) is recovered from the distillation facility. The light products can be used as a feedstock for ethylene production. A light diesel fuel is recovered which consists predominantly of normal paraffins (45). A heavy diesel fraction (46) is also recovered. The heavy diesel fraction is processed in a hydroisomerization dewaxing facility which converts the heavy normal paraffins into isoparaffins. The isomerized heavy diesel fuel (52) is blended with the light diesel fuel to produce a blended diesel (55) which meets the specification for cloud point and has a cetane index in excess of 60.

While this embodiment describes the production of diesel fuel, it is very similar to an equivalent embodiment for the production of jet fuel, with an adjustment in the product boiling ranges.

Upgrading of Waxy Petroleum Products with Lubricant Base Oil Production

This embodiment describes a preferred method to convert waxy petroleum products into a lubricant base oil with production of valuable normal paraffin rich by-products.

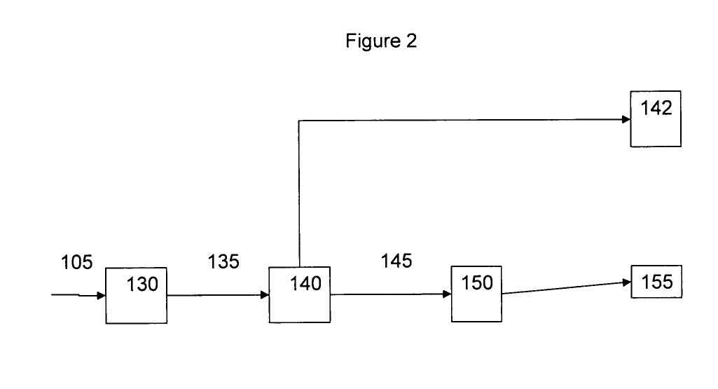

Lubricant base oils are made from petroleum products that contain 650° F.+ boiling range material. 650° F.+ containing waxy petroleum products, such as hydrotreated petroleum streams, hydrocracked petroleum streams, hydroisomerized petroleum streams, slack waxes, etc., can be processed in a hydroconversion reactor to selectively convert the normal paraffins in the feed to lighter normal paraffins. The lighter normal paraffins can be separated from the unconverted product by distillation. The lighter normal paraffins can then be used to make solvents, jet fuels, jet fuel blend components, diesel fuels, diesel fuel blend components, and feedstocks for the production of linear alkyl benzenes. The unconverted product, now depleted at least in part of normal paraffins, can be converted into a lubricant base oil by processes comprising solvent dewaxing, catalytic dewaxing, hydrotreating, hydrocracking, solvent extraction, and combinations. Catalytic dewaxing is preferred, especially hydroisomerization dewaxing. In hydroisomerization dewaxing, waxy materials are catalytically isomerized to lower their pour point. The selectivity of normal paraffins during this isomerization is lower than the selectivity of other compounds. The relatively poorer selectivity of the normal paraffins during this operation results in the production of less valuable lighter products which typically include mixtures of normal and isoparaffins. By selectively converting the normal paraffins in the feed to valuable normal paraffin lighter products, the overall economics are improved. In FIG. 2, a waxy 650° F.+ petroleum feedstock (105), preferably a slack wax, is processed in a hydroconversion reactor (130). The hydroconversion reactor uses a 10-ring borosilicate, preferably silica-bound Pt/B-ZSM-5 containing less than 20 weight ppm aluminum, and operates at 250 psig, 1 LHSV, and at a temperature to give 70 percent per pass conversion. The effluent from the hydrocracker (135) is also sent to the distillation facility (140). A light product, or series of light products, (142) is recovered from the distillation facility. The light products can be used as a feedstock for ethylene production, jet fuel, diesel fuel, or linear alkyl benzenes. A 650° F.+ unconverted portion (145) is withdrawn from the distillation facility. It is processed in a lubricant base oil manufacturing facility (150) to produce a lubricant base oil (155). Preferably the lubricant base oil manufacturing facility comprises a catalytic hydroisomerization reactor that uses Pt on a 10-ring molecular sieve to isomerize the residual waxy species in the unconverted product and lower the pour point to the desired value. Most preferably the molecular sieve used in the hydroisomerization reactor is SAPO-11, ZSM-23, or SSZ-32. The conditions for the hydroisomerization reactor are 1000 psig, 1 LHSV, 5000 SCFB, and at a temperature to achieve the desired product pour point. The lubricant base oil manufacturing facility also comprises a distillation section to adjust the viscosity and flash of the base oil products, and a hydrotreater to reduce the content of aromatics and improve color and stability.

What is claimed is:

1. A process for converting a hydrocarbonaceous feed containing greater than 5 wt. % C₁₀₊ n-paraffins in the presence of hydrogen to produce n-paraffin products lower in molecular weight than the C₁₀₊ n-paraffins in the feed by contacting the feed under conditions comprising:
a. temperature between 600 and 800° F.,
b. pressure between 50 and 5000 psig,
c. LHSV between 0.5 and 5
with a catalyst comprising a (1) borosilicate or aluminoborosilicate molecular sieve containing at least 0.05 weight percent boron and less than 1000 ppm by weight of aluminum, or a titanosilicate molecular sieve and (2) a Group 8 metal.

2. The process according to claim 1 wherein the borosilicate or aluminoborosilicate molecular sieve contains less than 200 ppm by weight of aluminum.

3. The process according to claim 1 wherein the borosilicate or aluminoborosilicate molecular sieve contains less than 20 ppm by weight of aluminum.

4. The process according to claim 1 wherein the C₆ product has an iso/normal weight ratio of less than 0.2.

5. The process according to claim 1 wherein the C₆ product has an iso/normal weight ratio of less than 0.05.

6. The process according to claim 1 wherein the C₆ product has an iso/normal weight ratio of less than 0.01.

7. The process according to claim 1 wherein the product further comprises a C₁₃ product having an iso/normal weight ratio less than 2.

8. The process according to claim 7 wherein the C₁₃ product has an iso/normal weight ratio of less than 0.5.

9. The process according to claim 7 wherein the C₁₃ product has an iso/normal weight ratio of less than 0.1.

10. The process according to claim 1 wherein the Group 8 metal is selected from the group consisting of Pt, Pd, Rh, Ir, Ru, Os and combinations thereof.

11. The process according to claim 10 wherein the at Group 8 metal is Pt.

12. The process according to claim 10 wherein the amount of metal is between 0.1 and 5 wt % based on the weight of the molecular sieve.

13. The process according to claim 10 wherein the amount of metal is between 0.1 and 3 wt % based on the weight of the molecular sieve.

14. The process according to claim 10 wherein the amount of metal is between 0.3 and 1.5 wt % based on the weight of the molecular sieve.

15. The process according to claim 1 wherein the molecular sieve is a zeolite.

16. The process according to claim 15 wherein the zeolite contains pores less than or equal to 12 rings and a dimensionality greater than or equal to 1.

17. The process according to claim 16 wherein the pores are less than or equal to 10 rings and the dimensionality is greater than or equal to 2.

18. The process according to claim 16 wherein the dimensionality of the pores is greater than or equal to 3.

19. The process according to claim 15 wherein the zeolite is selected from the group consisting of SSZ-13, SSZ-33, SSZ-46, SSZ-53, SSZ-55, SSZ-57, SSZ-58, SSZ-59, SSZ-60, SSZ-64, SSZ-70, ZSM-5, ZSM-11, TS-1, MTT and H-Y.

20. The process according to claim 19 wherein the zeolite is ZSM-5.

21. The process according to claim 1 wherein the feed is selected from the group consisting of slack wax, Fischer Tropsch products, and combinations thereof.

22. The process of claim 1 the hydrocarbonaceous feed contains less than 100 ppm each of sulfur and nitrogen.

23. The process of claim 1 wherein the catalyst has been exposed to sulfur prior to contact with the n-paraffin-containing hydrocarbonaceous feed.

24. A process for converting a hydrocarbonaceous feed containing greater than 5 wt. % C₁₀₊ n-paraffins in the presence of hydrogen to produce n-paraffin products lower in molecular weight than the C₁₀₊ n-paraffins in the feed by contacting the feed under conditions comprising:
a. temperature between 600 and 800° F.,
b. pressure between 50 and 5000 psig,
c. LHSV between 0.5 and 5
d. conversion of n-paraffins in the feed to smaller n-paraffins of 25% to 99%
with a catalyst comprising (1) a borosilicate or aluminoborosilicate molecular sieve containing at least 0.05 weight percent boron and less than 1000 ppm by weight of aluminum, or a titanosilicate molecular sieve and (2) a Group 8 metal wherein the selectivity for the conversion of n-paraffins in the feed to smaller n-paraffins is equal to or greater than 60%.

25. The process of claim 24 wherein the selectivity is equal to or greater than 80%.

26. The process of claim 24 wherein the selectivity is equal to or greater than 90%.

27. The process of claim 24 wherein the selectivity is equal to or greater than 95%.

28. The process according to claim 24 wherein the borosilicate or aluminoborosilicate molecular sieve contains less than 200 ppm by weight of aluminum.

29. The process according to claim 24 wherein the borosilicate or aluminoborosilicate molecular sieve contains less than 20 ppm by weight of aluminum.

30. The process according to claim 24 wherein the C₆ product has an iso/normal weight ratio of less than 0.2.

31. The process according to claim 24 wherein the C₆ product has an iso/normal weight ratio of less than 0.05.

32. The process according to claim 24 wherein the C₆ product has an iso/normal weight ratio of less than 0.01.

33. The process according to claim 24 wherein the product further comprises a C₁₃ product having an iso/normal weight ratio less than 2.

34. The process according to claim 33 wherein the C₁₃ product has an iso/normal weight ratio of less than 0.5.

35. The process according to claim 33 wherein the C₁₃ product has an iso/normal weight ratio of less than 0.1.

36. The process according to claim 24 wherein the Group 8 metal is selected from the group consisting of Pt, Pd, Rh, Ir, Ru, Os and combinations thereof.

37. The process according to claim 36 wherein the at Group 8 metal is Pt.

38. The process according to claim 36 wherein the amount of metal is between 0.1 and 5 wt % based on the weight of the molecular sieve.

39. The process according to claim 36 wherein the amount of metal is between 0.1 and 3 wt % based on the weight of the molecular sieve.

40. The process according to claim 36 wherein the amount of metal is between 0.3 and 1.5 wt % based on the weight of the molecular sieve.

41. The process according to claim 24 wherein the molecular sieve is a zeolite.

42. The process according to claim 41 wherein the zeolite contains pores less than or equal to 12 rings and a dimensionality greater than or equal to 1.

43. The process according to claim 42 wherein the pores are less than or equal to 10 rings and the dimensionality is greater than or equal to 2.

44. The process according to claim 42 wherein the dimensionality of the pores is greater than or equal to 3.

45. The process according to claim 41 wherein the zeolite is selected from the group consisting of SSZ-13, SSZ-33, SSZ-46, SSZ-53, SSZ-55, SSZ-57, SSZ-58, SSZ-59, SSZ-60, SSZ-64, SSZ-70, ZSM-5, ZSM-11, TS-1, MTT and H-Y.

46. The process according to claim 45 wherein the zeolite is ZSM-5.

47. The process according to claim 24 wherein the feed is selected from the group consisting of slack wax, Fischer Tropsch products, and combinations thereof.

48. The process of claim 24 wherein the hydrocarbonaceous feed contains less than 100 ppm each of sulfur and nitrogen.

49. The process of claim 24 wherein the catalyst has been exposed to sulfur prior to contact with the n-paraffin-containing hydrocarbonaceous feed.
